(19) United States
(12) Patent Application Publication
(10) Pub. No.: US 2023/0416798 A1
(43) Pub. Date: Dec. 28, 2023

(54) METHODS OF RECOMBINANT ANTIBODY PRODUCTION AND RELATED CELL SYSTEMS

(71) Applicant: MBrace Therapeutics, Inc., San Diego, CA (US)

(72) Inventors: Fernanda I. Staquicini, Jersey City, NJ (US); Renata Pasqualini, Newark, NJ (US)

(73) Assignee: MBrace Therapeutics, Inc., San Diego, CA (US)

(21) Appl. No.: 18/311,224

(22) Filed: May 2, 2023

Related U.S. Application Data
(60) Provisional application No. 63/337,980, filed on May 3, 2022.
(60) Provisional application No. 63/359,541, filed on Jul. 8, 2022.

(51) Int. Cl. C12P 21/02; C12N 5/071; C12N 15/11; C12N 9/22; C12N 15/90; C12N 15/10; C07K 16/18; C07K 16/40; C07K 16/28

(52) U.S. Cl. CPC C12P 21/02; C12N 5/0686; C12N 15/11; C12N 9/22; C12N 15/907; C12N 15/102; C07K 16/18; C07K 16/40; C07K 16/2818; C12N 2501/998; C12N 2310/20; C12N 2800/80; C07K 2317/24

(57) ABSTRACT

Provided herein are methods and cell systems for production of a recombinant antibody in cells in which the antibody is directed against a target protein that is natively expressed as an intracellular protein in the cell. Also provided herein are modified cell lines for use in such methods in which the modified cell line is engineered to express a variant target protein that has a mutant epitope of the target epitope of an antibody.

### RELATED APPLICATIONS

This application claims priority to U.S. Provisional Patent Application Ser. No. 63/337,980 filed on May 3, 2022 and U.S. Provisional Patent Application Ser. No. 63/359,541 filed on Jul. 8, 2022, each of which are hereby incorporated by reference into this application in their entirety.

### INCORPORATION OF SEQUENCE LISTING

A computer readable form of the Sequence Listing is filed with this application by electronic submission and is incorporated into this application by reference in its entirety. The Sequence Listing is contained in the file created on May 2, 2023, having the file name MBRACE002.xml, and is 35,971 bytes in size (as measured in the MS-Windows® operating system).

### FIELD

The present disclosure relates to methods and cell systems for production of a recombinant antibody in cell lines in which the antibody is directed against a target protein that is natively expressed as an intracellular protein in the cell line. The present disclosure also provides modified cell lines for use in such methods in which the modified cell line is engineered with a variant target protein that has a mutant epitope of the target epitope of an antibody.

### BACKGROUND

Engineered antibody molecules and their fragments are being increasingly exploited as scientific and clinical tools for therapy and diagnosis of diseases. The unique ability of antibodies to specifically recognize and bind with high affinity to virtually any type of antigen makes them attractive as a starting point for novel biopharmaceutical products and scientific research. Recently certain stress proteins normally present as intracellular proteins have been identified as unique targets for antibody-based therapies in cancer and other pathological conditions since such proteins generally are not expressed, or are expressed at low levels, on the surface of normal cells. Improved methods are needed for producing such antibodies in cells, including antibodies and their fragments that specifically recognize and bind intracellular targets in mammalian cells.

### SUMMARY OF ILLUSTRATIVE EMBODIMENTS

The foregoing general description of the illustrative implementations and the following detailed description thereof are merely exemplary aspects of the teachings of this disclosure, and are not restrictive.

The present disclosure provides, in selected embodiments, methods and cell lines for the manufacture of antibodies that bind to intracellular target proteins. The present disclosure also provides methods of modifying cell lines for the manufacture of antibodies that bind to intracellular target proteins.

In some embodiments, the disclosure provides methods of recombinant production of an antibody comprising expressing a nucleic acid encoding an antibody in a modified cell line, wherein the antibody specifically binds to a target epitope on a target protein that is natively expressed as an intracellular protein in the cells and wherein the modified cell is engineered to express a variant target protein that comprises an alteration of one or more amino acid residues of the target epitope, and culturing the modified cell line under conditions so that the antibody is produced.

In some embodiments, the disclosure provides methods of recombinant production of an antibody comprising expressing a nucleic acid encoding an antibody in a modified mammalian cell line, wherein the antibody specifically binds a target epitope on a target protein that is natively expressed as an intracellular protein in the mammalian cell and wherein the modified mammalian cell is engineered to express with a variant target protein that comprises an alteration of one or more amino acid residues of the target epitope, and culturing the modified mammalian cell line under conditions so that the antibody is produced.

In some embodiments, the target protein that is natively expressed is an intracellular protein in the secretory pathway of the cell line. In some embodiments, the target protein is a stress protein.

In some embodiments, the target protein that is natively expressed as an intracellular protein in the cell is modified with an inert mutation that retains the structure and function of the target protein in the cell. In some embodiments, target protein that is natively expressed as an intracellular protein in the cell is modified with one or more mutations that reduce or abrogate binding of the antibody to the target epitope on the native target protein.

In some embodiments, the target protein is associated with a membrane within the cell. In some embodiments the membrane is an organelle membrane. In some embodiments the membrane is the plasma membrane.

In some embodiments, the target protein is an intracellular signaling protein, such as those involved in a signal transduction pathway, e.g., a kinase or a phosphatase.

In some embodiments, the target protein is expressed on an organelle of the cell line. In specific embodiments, the target protein is expressed on the endoplasmic reticulum. In specific embodiments, the target protein is expressed on the Golgi apparatus.

In some embodiments, the target protein is an endoplasmic reticulum protein, e.g., calreticulin, a heat shock protein or an isomerase. In specific embodiments, the target protein is an endoplasmic reticulum chaperone, e.g., glucose-regulated protein 78 (GRP78), HSP47, protein disulphide isomerase (PDI), calreticulin or GP94.

In some embodiments, the target protein is a Golgi complex protein, e.g., Golgi phosphoprotein 2 (GOLPH2), Golgi phosphoprotein 3 (GOLPH3), GM130, ATPase H+ Transporting V1 Subunit A (ATP6V1A), ATPase H+ Transporting V1 Subunit E1 (ATPP6V1E1), ATPase H+ Transporting V0 Subunit A2 (ATP6VOA2), transmembrane protein 165 (TMEM165), Golgin B1 (GOLGB1), SCY1-like 1-binding protein 1 (SCYL1BP1), Trafficking Protein Particle Complex Subunit 11 (TRAPPC11), Trafficking Protein Particle Complex Subunit 2 (TRAPPC2), or Thyroid Hormone Receptor Interactor 11 (TRIP11).

In some embodiments the disclosure provides methods of recombinant production of an antibody targeting an epitope on a target found intracellularly in a cell line, the method comprising providing an antibody that selectively binds to an epitope on a target protein that is present intracellularly in a cell line, identifying the epitope of the target protein to which the antibody binds, modifying the epitope of the target protein in the cell line to reduce or abrogate the binding of the antibody to the epitope on the target protein, introducing an expression vector encoding the antibody to the modified cell line, and culturing the modified cell line under conditions that allow production of the antibody from the expression vector in the modified cell line.

In some embodiments the disclosure provides methods of recombinant production of an antibody targeting an epitope on a target found intracellularly in a mammalian cell line, the method comprising providing an antibody that selectively binds to an epitope on a target protein that is present intracellularly in a mammalian cell line, identifying the epitope of the target protein to which the antibody binds, modifying the epitope of the target protein in the mammalian cell line to reduce or abrogate the binding of the antibody to the epitope on the target protein, introducing an expression vector encoding the antibody to the modified mammalian cell line, culturing the modified mammalian cell line under conditions that allow production of the antibody from the expression vector, and expressing a nucleic acid encoding an antibody in the modified mammalian cell line.

Examples of mammalian cell lines that can be used with the methods of the present disclosure include Chinese hamster ovary (CHO) cells, baby hamster kidney cells, NSO myeloma cells, monkey kidney COS cells, monkey kidney fibroblast CV-I cells, human embryonic kidney 293 (HEK293) cells, human breast cancer SKBR3 cells, human leukemia Jurkat T cells, dog kidney MDCK cells, and human cervical cancer HeLa cells.

In some embodiments, the methods further comprise isolating the antibody, e.g., from the culture supernatant.

In some embodiments the disclosure provides methods of expressing a nucleic acid encoding an antibody in a modified mammalian cell line, wherein the antibody selectively binds to GRP78 and wherein the modified mammalian cell is engineered with a variant GRP78 that has a mutant epitope comprising a mutation of one or more amino acid residues of the target epitope of the antibody or antigen binding fragment, and culturing the modified mammalian cell line under conditions so that the anti-GRP78 antibody is produced. In some embodiments, the methods further comprise isolating the anti-GRP78 antibody, e.g. from the culture supernatant.

The antibody produced using the methods of the disclosure can include human antibodies and humanized antibodies. In some embodiments the antibody is a full-length antibody. In specific embodiments, the antibody is an antigen-binding fragment. In some embodiments, the antibody is an IgG, e.g., IgG1.

The exemplary embodiments can employ various methods for modifying cell lines, as will be apparent to one skilled in the art upon reading the present disclosure. In some embodiments, the mutations to the native target protein can be introduced using nuclease-based editing techniques, e.g., using a nuclease selected from meganucleases, Zinc finger nucleases (ZFNs), transcription-activator like effector nucleases (TALENs) and an RNA-guided nuclease. In some embodiments, the target protein is modified using nuclease-mediated homology-directed repair (HDR), homologous recombination (HR) or a combination thereof.

In some embodiments, the cell line is modified using RNA-directed nucleases. Such editing systems employ an RNA-guided nuclease, a guide RNA, and a homology-directed repair (HDR) template (i.e., a donor template), wherein the HDR template is homologous to the target protein and comprises nucleotides encoding the mutant epitope of the target protein. In specific embodiments, the RNA-guided nuclease is a Type II or Type V RGN.

In some embodiments, the disclosure provides methods of making a modified mammalian cell line for recombinant antibody production comprising introducing into a mammalian cell line a DNA-targeting nuclease for inducing a genetic disruption of a target site within a gene encoding a target protein, wherein the target protein is a protein that is natively expressed as an intracellular protein in the mammalian cell line and introducing into the mammalian cell line a template polynucleotide, wherein the template polynucleotide is homologous to the gene encoding the target protein and comprise nucleotides encoding a mutant epitope comprising a mutation of one or more amino acid residue of a target epitope present in the target protein that is specifically bound by an antibody or antigen binding fragment, wherein the template polynucleotide is targeted for integration at or near the at least one target site via nuclease-mediated homology directed repair (HDR) and after the integration of the template polynucleotide into the gene, the gene encodes a variant target protein comprising the mutant epitope.

In some embodiments, the disclosure provides a modified mammalian cell line comprising a variant target protein that is an intracellular protein. The variant target protein comprises a mutation of a native intracellular protein that contains a target epitope that an antibody selectively binds, and the mutation of the variant target protein is an amino acid substitution of one or more amino acid residues to change the target epitope of the antibody to a mutant epitope.

Other features, advantages, and aspects will be described below in more detail.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values or dimensions illustrated in the accompanying graphs and figures are for illustration purposes only and may or may not represent actual or preferred values or dimensions. Where applicable, some or all features may not be illustrated to assist in the description of underlying features.

FIG. 1 depicts SDS-PAGE analysis of purified supernatant samples following expression of anti-GRP78 antibody clones B4, D1 and F6 in CHO cells under reducing and non-reducing conditions.

FIG. 2A and FIG. 2B depict flow cytometry results for binding of antibody clones B4 and F6, respectively, to GRP78 variants from an alanine scan library. For each GRP78 variant, the percent mean fluorescence for WT GRP78 binding is shown plotted relative to the control antibody 1H11-1H7.

FIG. 3 depicts visualization of major residues for antibody binding of anti-GRP78 clones B4 and F6 to GRP78, with exemplary major and other residues involved in binding shown by arrows.

FIG. 4 depicts genomic sequence analysis of interacting residues for anti-GRP78 clones B4 and F6 for residues in GRP78 located in Exon 5 and flanking intron residues. The amino acid sequence of the full length GRP78 is also shown.

FIG. 5A depicts the design of dual gRNA and ssDNA donor template for introducing exemplary mutations into GRP78 by HDR-based CRISPR gene editing.

FIG. 5B depicts design of primer pairs for PCR amplification used to validate gene editing shown in FIG. 5A.

### DETAILED DESCRIPTION

The description set forth below in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

It must be noted that, as used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “about,” “proximate,” “minor variation,” and similar terms generally refer to ranges that include the identified value within a margin of 20%, 10% or preferably 5% in certain embodiments, and any values therebetween.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

Provided herein are methods of recombinant production of antibodies or binding fragments thereof in cells in which the target protein of the antibody is also found as an intracellular protein in the cells. In some embodiments, the cells in which the antibodies or binding fragments are produced are mammalian cells. In some embodiments, the target intracellular protein to which the antibody binds is a protein involved in the secretory pathway, such as proteins expressed on or in the endoplasmic reticulum (ER) or Golgi apparatus. In some embodiments, the target protein of the antibody is a stress protein, which are proteins that normally function in the ER or Golgi apparatus or other intracellular organelle but that may become upregulated on the surface of cells under conditions of cell stress. For instance, stress proteins, such as those involved in the unfolded protein response (UFR), are proteins found to be upregulated on the surface of cancer cells but not normally found on the surface of normal cells. Stress proteins exhibit desirable features for use as a target protein for antibody-based cancer therapies since such proteins generally are not expressed or are expressed at low levels on the surface of normal cells, whereas they can become upregulated or overexpressed at the surface of cancer cells. The use of stress proteins as a target protein of an antibody-based therapy ensures that their upregulated surface expression in a variety of cancers, but not in normal tissue and cells, and reduces or minimizes off-target activity and/or toxicity.

Methods to produce antibodies in sufficient quantities for use as a therapeutic are required. Several expression systems are available, both from prokaryotic and eukaryotic origin. The choice of system depends on many factors, including the molecular species being expressed and the precise sequence of the individual antibody.

Particularly desirable for recombinant production of human or humanized antibodies are mammalian expression systems. For instance, mammalian cells are able to carry out desired protein folding and post-translational modifications identical to human systems. By expression of recombinant proteins in mammalian cells, such as HEK293 cells or CHO cells, it is possible to achieve a glycosylation pattern which is similar but not identical to that obtained from human cells. Mammalian cells allow for very high product yields and are comparatively robust to metabolic stresses.

Although efforts have been made to produce antibodies to stress proteins, it has not been possible to generate them efficiently in mammalian cells. For example, observations herein demonstrate that antibodies directed against the exemplary stress protein GRP78 are not able to be expressed in high yield from mammalian cells without the using the methods of the present disclosure. Without wishing to be bound by theory, it is believed that, in certain circumstances, stress proteins are required for cell viability or function of the mammalian cell, such that binding of the intracellular protein by an antibody during the course of recombinant antibody production is detrimental to the cell. For example, in some embodiments, binding of certain intracellular proteins inhibits the ability of the intracellular protein to carry out its normal function in the cell, leading to misfolding of proteins in cells, lack of degradation of misfolded proteins, improper calcium homeostasis and, in some cases, a reduction or loss of cell viability. This loss or decrease of function impacts the ability to produce antibody in high yield.

Studies have demonstrated that stress proteins, such as chaperones, are necessary to function properly in a cell during antibody production. For instance, changes in cellular abundance of secretory pathway proteins have been shown to correlate with the ability to produce recombinant monoclonal antibodies abundantly (see e.g., Lambert and Merten, 1997, Biotechnol. Bioeng., 54:165-180; Downham et al. 1996, Biotechnol. Bioeng., 51:691-696). It is known that secretory proteins are folded and assembled into higher order complexes in the cell's inner endoplasmic reticulum (ER) compartment of the cell shortly after protein synthesis. The ER is composed of specific auxiliary assembly factors along with quality control mechanism (Ellgaard et al., Quality control in the secretory pathway, Science. 1999 Dec. 3; 286: 1882-8; Helenius et al., Intracellular functions of N-linked glycans, Science. 2001 Mar. 23; 291:2364-9). The folding of proteins and the refolding of misfolded soluble and aggregated proteins is known to be mediated by a network of evolutionarily conserved protein molecules called chaperones (Haiti, F. U., Nature, 381, 571-580, (1996); Horwich, A. L., Brooks Low K., Fenton, W. A., Hirshfield, I. N. & Furtak, K., Cell 74, 909-917 (1993); Ellis, R. J. & Hemmingsen, S. M., TiBS, 14, 339-342, (1989); Bukau, B., Hesterkamp, T. & Luirink, J., Trends Cell Biol, 6, 480-486, (1996); Bukau, B., Deuerling, E., Pfund, C. & Craig, E. A., Cell, 101, 119-122, (2000)). Secretory proteins also undergo various post-translational modifications, including glycosylation, during their passage through the Golgi complex.

Alternatively, even if the binding of an intracellular protein by an antibody does not impact cell viability, it can impact the manufacture of the antibody due to sequestration of the product by the intracellular protein. Briefly, the intracellular protein can act as a “sink” for the binding of the antibody or antigen-binding fragment, preventing secretion and thus limiting the yield of the intended production.

The provided methods relate to modified cell lines for production of antibodies or antigen-binding fragments that target an endogenous intracellular target protein in a cell, such as a target protein in the secretory pathway. In some embodiments, the cells are mammalian cells. In some embodiments, the target protein is a stress protein. In some embodiments, the provided methods involve producing a modified mammalian cell carrying a variant of the target protein by mutation of the target epitope to a mutant epitope, such that the binding of the antibody to the endogenous target protein in the mammalian cell is reduced or abrogated. In particular embodiments, the mutation is a genomic mutation of the target protein that is introduced by a gene editing method, such as by nuclease-mediated gene editing. In some embodiments, the mutation is introduced using an RNA-guided nuclease, zinc-finger nuclease, meganuclease or transcription activator-like effector nuclease. In some cases, the mutation is introduced by homology-directed repair (HDR). In particular embodiments, the mutation is an inert mutation such that the variant target protein retains the structure and function of the endogenous target protein but the epitope of the antibody is modified to abrogate binding of the antibody to the target protein. In the provided methods, the modified cells can then be used for production of the antibody or antigen-binding fragment, such as by expressing a nucleic acid molecule encoding the antibody in the cells and culturing the cell under conditions to produce the antibody. In some embodiments, the produced antibody can be isolated from the cell.

Results herein demonstrate that the provided methods can substantially improve protein production in mammalian cells. In some embodiments, the amount of antibody produced from modified mammalian cells in accord with the provided methods is increased greater than 2-fold or more compared to production of the same antibody in the native mammalian cell in which the target protein is not mutated. In some embodiments, the improvement in antibody production is by greater than at or about 5-fold, 10-fold, 15-fold, 20-fold, 30-fold, 40-fold, 50-fold or more compared to production of the same antibody in the native mammalian cell in which the target protein is not mutated. For instance, in some cases, the improvement in antibody production is by at least 20-fold compared to the native mammalian cell in which the target protein is not mutated.

The provided cell systems and methods are exemplified for antibodies targeting GRP78 and other intracellular target proteins. The cell systems and methods described herein are generalizable to production of all or part of an antibody-based biologic that selectively targets proteins that are generally found intracellularly, but which can be found on the cell surface in certain pathologies. It will thus be understood by those of ordinary skill in the art that the present disclosure is exemplary and can be applied to other cell systems and manufacturing methods for production of biologics targeting such other targets.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### Methods of Antibody Production

Provided herein is a method of recombinant production of an antibody in which the antibody binds to a target protein (or a homolog thereof) that is normally expressed natively as an intracellular protein. In the provided cell or cell-free systems and methods, the antibody is produced in a modified system, e.g., a mammalian cell line or cell-free system, that is genetically altered to contain a variant of the target protein in which binding of the antibody to such variant target protein is reduced or abrogated. In some embodiments, the variant target protein is modified in a mammalian cell line used to make the system so that it contains a mutant epitope with a mutation of one or more amino acid residues of the target epitope of the antibody to reduce or abrogate recognition by the antibody or antigen-binding fragment. In particular embodiments, the mutation of the variant target protein with a mutant epitope is an inert mutation that retains the structure and function of the native intracellular protein, thereby ensuring function of the target protein. Provided methods and systems using the modified cell lines or cell-free systems thus allow for high yield production of the antibody in a manufacturing system. These methods and systems disclosed herein can be used individually or in conjunction with all or specific aspects of other antibody manufacturing systems. For instance, in some preferred embodiments the methods can utilize aspects of cell-free systems disclosed in U.S. Ser. No. 63/359,871 filed on Jul. 10, 2022 and/or U.S. Ser. No. 63/398,143 filed on Aug. 15, 2022, each of which are hereby incorporated into this application in their entirety for all purposes.

In some preferred embodiments, the cell-free systems of U.S. Ser. No. 63/359,871 filed on Jul. 10, 2022 can comprise methods of recombinant production of an antibody by: a) expressing a nucleic acid encoding an antibody in a modified cell-free system (in some preferred embodiments a eukaryotic cell-free system), wherein the antibody specifically binds to a target epitope on a protein of the system, and wherein the modified cell-free comprises a mutation of one or more amino acid residues of the target epitope on the protein in the system; and, initiating transcription and translation of the antibody in the cell-free system under conditions so that the antibody is produced. In some preferred embodiments, such a cell-free system can comprise a cell lysate (in some preferred embodiments a eukaryotic cell lysate). In some preferred embodiments, the cell-free systems can comprise: a) modifying a cell line (in some preferred embodiments, a mammalian cell line) to abrogate binding of an antibody to an intracellular protein in the cells of the cell line, wherein the modification results in expression of a variant target protein that comprises a mutation of one or more amino acid residues of the target epitope of the antibody; b) creating a cell-free antibody production system from the modified cell line; c) introducing a nucleic acid template to the cell-free antibody production system, wherein the nucleic acid template encodes the antibody to the target protein; and, d) initiating transcription and translation from the nucleic acid template to produce the antibody in the cell-free antibody production system (and, in some preferred embodiments, isolating the antibody).

In some preferred embodiments, the cell-free systems of U.S. Ser. No. 63/398,143 filed on Aug. 15, 2022 can comprise: a) providing a cell-free protein expression system (in some preferred embodiments a mammalian cell-free system) containing a target protein exhibiting an antibody epitope; b) modifying the cell-free system by introducing one or more agents that block the antibody epitope (or in some preferred embodiments and antibody paratope on the target protein) on the target protein but do not eliminate the activity of the target protein in the cell-free system; c) introducing one or more nucleic acids encoding an antibody that binds to the antibody epitope to the cell free system; and, d) initiating transcription and translation of the antibody in the cell-free system under conditions so that the antibody is produced. In some preferred embodiments, the cell-free systems can comprise: a) introducing an agent that selectively binds to a target epitope on an intracellular protein to cells (in some preferred embodiments mammalian cells) to create a modified cell line; b) creating a cell-free antibody production system from the modified cell line; c) introducing a nucleic acid template to the cell-free antibody production system, wherein the nucleic acid template encodes an antibody that selectively binds to the target epitope on the intracellular protein; and, d) initiating transcription and translation from the nucleic acid template to produce the antibody in the cell-free antibody production system (and, in some preferred embodiments, isolating the antibody).

In some embodiments, the provided methods involve (a) expressing a nucleic acid encoding an antibody in a modified cell line, wherein the antibody is directed against a target protein or a homolog of the target protein that is natively expressed as an intracellular protein in the cell and wherein the modified cell is engineered with a variant target protein that has a mutant epitope comprising a mutation of one or more amino acid residue of the target epitope of the antibody or antigen binding fragment; and (b) culturing the modified cell line under conditions so that the antibody is produced. In some embodiments, the methods may further include isolating the antibody from the culture supernatant.

In some embodiments, the provided methods involve (a) expressing a nucleic acid encoding an antibody in a modified mammalian cell line, wherein the antibody is directed against a target protein or a homolog of the target protein that is natively expressed as an intracellular protein in the mammalian cell and wherein the modified mammalian cell is engineered with a variant target protein that has a mutant epitope comprising a mutation of one or more amino acid residue of the target epitope of the antibody or antigen binding fragment; and (b) culturing the modified mammalian cell line under conditions so that the antibody is produced. In some embodiments, the methods may further include isolating the antibody, e.g., from the culture supernatant.

### Intracellular Protein Targets and Antibodies to Same

In some embodiments, the provided cell systems and methods are useful for expressing antibodies in cells, such as mammalian cells, in which the target protein of the antibody is natively expressed as an intracellular protein in the cell. In particular embodiments, the target protein is a protein that is aberrantly expressed on the surface of cells in cancer and other diseases but that also is necessary for the function of normal cells due to the role of the target protein in normal intracellular biological processes. In some embodiments, the target protein is a protein that is involved in the secretory pathway of the cell, such as in a mammalian cell line. In some embodiments, the target protein is expressed on an organelle of the cell, such as in a mammalian cell line. In some embodiments, the target protein is expressed on or in the endoplasmic reticulum. In some embodiments, the target protein is expressed on or in the Golgi apparatus.

In some embodiments, the intracellular protein target is a stress protein. Stress proteins include proteins that are involved in organelle autoregulation to maintain homeostasis to regulate the capacity of the organelle under stress conditions, such as may occur in a tumor environment. Various stress proteins are known and include proteins in the secretory pathway. Various stress proteins, including those resulting from ER stress or Golgi stress, are known: Sasaki and Yoshida, J Biochem. 157:185, 2015; Sasaki and Yoshida, FEBS Letters, 593:2330-2340, 2019; Gao et al., Biofactors, 47:964-974, 2021; Li et al., Mol Neurobiol, 49:1449-59, 2014; Yadav et al., J Cancer Prev., 19:75-88, 2014; and Chen and Cubillos-Ruiz, Nature Reviews Cancer, 21:71-88, 2021.

For example, targets include endoplasmic reticulum chaperones (e.g., calreticulin, heat shock proteins, and isomerases) that can translocate to the cytosol and eventually the surface of cells, particularly during under stress conditions. These proteins have been found to be overexpressed in certain pathological conditions. See, e.g., Weirsma V R et al., Front. Oncol., Vol 5: Art. 7 (2015); Garg A D, Cancer Immunol Immunother 61:215-21 (2012). The disclosure of the present embodiments and application of the teachings therein allow production of biologics targeting endoplasmic reticulum chaperones, as well as other identified targets, without overly compromising cell viability. Exemplary biologics can include, for example, those targeting HSP47 for treatment of cervical cancer, stomach cancers or autoimmune disease (Yokota S et al., Biochem Biophys Res Commun 303:413-8 (2003); Yamamoto N et al., Int J Oncol 43:1855-63 (2013)); biologics targeting protein disulfide isomerase (PDI) for the treatment of central nervous system cancers, ovarian cancer, brain cancer, prostate cancer and lung cancer (Xu Set al., Free Radic Biol Med 52:993-1002 (2012); Zhang L et al., Cancer Invest 27:453-(2009); Pan Z et al., Int J Oncol 35:823-8 (2009)); and biologics targeting calreticulin for treatment of various cancers (Zamanian M et al., Pathol Oncol Res 149-54 (2013); Gold L I et al., FASEB J 24:665-83 (2010), biologics targeting GP94 (Marzec M. et al., Biochim Biophys Acta. 2012 March; 1823(3): 774-787), and biologics targeting GRP78 (Arap et al. Cancer Cell 6:275-284 (2004); Sato et al. Adv Genet 69:97-114 (2010).

A variety of Golgi complex proteins are known to be involved in various pathologic states, especially cancers and autoimmune disease, and monoclonal antibodies that selectively bind to these organelle proteins are suited for production of monoclonal antibodies using the methods as disclosed herein. Such proteins include, but are not limited to, Golgi phosphoprotein 2 (GOLPH2) (Liu et al., Front Oncol 2021 December 7; 11:78386); Golgi phosphoprotein 3 (GOLPH3) (Xing M, et al., Mol Biol Cell 27: 3828-3840, 2016; Scott K L, et al., Nature 459: 1085-1090, 2009); GM130 (Chang S H, et al., Mol Ther 20: 2052-2063, 2012); ATPase H+ Transporting V1 Subunit A (ATP6V1A) (Van Damme T, et al., Am J Hum Genet 100: 216-227, 2017); ATPase H+ Transporting V1 Subunit E1 (ATPP6V1E1) (Id.); ATPase H+ Transporting V0 Subunit A2 (ATP6VOA2) (Kornak U, et al., Nat Genet 40: 32-34, 2008); transmembrane protein 165 (TMEM165) (Rosnoblet C, et al., Hum Mol Genet 22: 2914-2928, 2013); Golgin B1 (GOLGB1) (Katayama K, et al., Biochem Biophys Res Commun 499: 459-465, 2018); SCY1-like 1-binding protein 1 (SCYL1BP1) (Hennies H C, et al, Nat Genet 40: 1410-1412, 2008); Trafficking Protein Particle Complex Subunit 11 (TRAPPC11) (Larson A A, et al, Skelet Muscle 8: 17, 2018); Trafficking Protein Particle Complex Subunit 2 (TRAPPC2) (Davis E E, et al., Clin Genet 85: 359-364, 2014); and Thyroid Hormone Receptor Interactor 11 (TRIP11) (Smits P, N Engl J Med 362: 206-216, 2010).

In some embodiments, the target protein is GRP78. In some embodiments, the target protein is heat shock protein 47 (HSP47). In some embodiments, the target protein is protein disulfide isomerase (PDI). In some embodiments, the target protein is calreticulin. In some embodiments, the target protein is GP94. In some embodiments, the target protein is GOLPH2. In some embodiments, the target protein is GOLPH3. In some embodiments, the target protein is GM130. In some embodiments, the target protein is ATP6V1A. In some embodiments, the target protein is AATPP6V1E1. In some embodiments, the target protein is ATP6VOA2. In some embodiments, the target protein is transmembrane protein 165 (TMEM165). In some embodiments, the target protein is GOLGB1. In some embodiments, the target protein is SCYL1BP1. In some embodiments, the target protein is TRAPPC11. In some embodiments, the target protein is Thyroid Hormone Receptor Interactor 11 (TRIP11).

A skilled artisan is familiar with antibodies to intracellular protein targets, such as stress proteins. For example, antibodies to GRP78 antibodies include: GRP78-specific mouse monoclonal IgG antibody MAb159 (Ojha and Amaravadi, Pharmacol. Res., 120:258-266, 2017), PAT-SM6 (Ojha and Amaravadi, 2017); anti-GRP78 antibodies described in PCT Publ. No. WO2018/057703, PCT Publ. No. WO2014/153056; PCT Publ. No. WO2008/105560; U.S. Publ. No. US2010/0041074; U.S. Pat. Nos. 10,259,884; and 10,851,161. Antibodies to protein disulfide isomerase (PDI) include, but are not limited to, Invitrogen PDI Monoclonal Antibody Clone 12 (Thermo Fisher Scientific Catalog #MA5-43389) or Invitrogen PDI Monoclonal Antibody clone 2F6G12H2 (Thermo Fisher Scientific Catalog #MA5-43389). Antibodies to calreticulin include, for example, mAb FMC 75 (Enzo Life Sciences, Cat. No. ADI-SPA-601) and mAb 16 (BD Transduction laboratories, Cat. No. 612137). Antibodies to GOLPH3 include, for example, Thermo Fisher Monoclonal Antibody Clone 905CT9.1.1 (Thermo Fisher Scientific Catalog #MA5-37626). Each of these antibodies can serve as a template for a humanized antibody, using methods such as those disclosed herein in more detail.

In some embodiments, the antibody is a human antibody. Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

In addition to using a non-human monoclonal antibody for humanization purposes, a non-human antibody can be used as a template for the selection of a fully human or near fully-human antibody. One particular method for selecting such human antibodies is the use of phage display technology. This methodology may use antibody screening techniques as described, e.g., in Dower et al., WO 91/17271 and McCafferty et al., WO 92/01047, U.S. Pat. Nos. 5,877,218, 5,871,907, 5,858,657, 5,837,242, 5,733,743 and 5,565,332. In these methods, libraries of phage are produced in which different antibodies are provided on the outer surfaces of the phage. The antibodies are usually displayed on the phage as Fv or Fab fragments. Antibodies with a desired specificity are selected by affinity to a selected epitope of the organelle protein.

In a particular exemplary method, human antibodies that selectively bind to an epitope of an organelle protein can be produced using the technique of Winter, WO 92/20791. In this method, either the heavy or light chain variable region of a non-human monoclonal antibody is used. If a light chain variable region is selected as the starting material, a phage library is constructed in which members display the light chain variable region of a non-human monoclonal antibody and a different heavy chain variable region. The heavy chain variable regions are obtained from a library of rearranged human heavy chain variable regions. A phage showing strong specific binding for an epitope of an organelle protein is selected. The human heavy chain variable region from this phage provides the basis for constructing an optimized phage library in which each phage displays the same heavy chain variable region identified from the first display library and a different light chain variable region. The light chain variable regions are obtained from a library of rearranged human variable light chain regions. Phage that display the variable regions of completely human antibodies that show strong specific binding for the epitope on the organelle protein are selected.

Among the provided antibodies are monoclonal antibodies, including monoclonal antibody fragments.

### Design of Mutant Epitope on the Target Protein

In some embodiments, the provided methods involve mutating a target protein to generate a variant target protein that contains a mutant epitope to abrogate or reduce binding by the antibody or antigen-binding fragment. In some embodiments, the mutant epitope is mutated by one or more amino acid residues changes of a target epitope of the antibody or antigen-binding fragment. Various methods can be used to map the epitope of the monoclonal antibody to be produced using the methods of the disclosure in order to determine the edits that can be made for production of such antibodies using the methods of the disclosure. Exemplary methods are taught in U.S. Pat. No. 11,174,479 to Greenleaf, et al. entitled “Devices and methods for display of encoded peptides, polypeptides, and proteins on DNA”, issued Nov. 16, 2021, and U.S. Pat. No. 11,061,036 to Wilson, et al., entitled “Epitope Mapping”, issued Jul. 13, 2021.

In some embodiments, residues in a target protein important for antibody binding can be mapped to define or identify the epitope or binding domain of the antibody, and subsequently mutated. In some embodiments, cDNA encoding the target protein is randomly mutated by methods such as by treatment with chemical mutagen, irradiation during replication, passage through error-inducing (mutator) cell lines or by oligonucleotide directed random mutagenesis. In some embodiments, oligonucleotide-directed random mutagenesis is used in which a preselected region of the protein is targeted for random mutagenesis. In some embodiments, the preselected region for random mutagenesis is one which is believed to contain a binding site or epitope. A preliminary assessment of a preselected region can be accomplished by any methods known in the art including hydropathicity analysis of the protein sequence or by crystal structure analysis.

In some embodiments, a peptide-based approach can be used to map an epitope of an antibody. In one approach, peptide display technologies, presenting a library of protein fragments on a microarray or the surface of E. coli or phage and finding out which ones the antibody binds to through microarray scanning or flow cytometry. In another approach, a series of overlapping peptides of the target protein or a preselected region thereof are screened for antibody binding, such as by ELISA or other technique to monitor binding interactions.

In some embodiments, the epitope can be predicted computationally. In some embodiments, the epitope is predicted using the PEASE tool (Sela-Culang, J. et al. (2014) Structure 22(4):646-57; Sela-Culang et al. (2015) Bioinformatics 31(8):1313-5). In some embodiments, the predicted epitope is validated experimentally. Methods of experimentally testing antibody epitopes are known in the art, including, for example, validation is performed using an antibody cross blocking assay, by performing an assay comprising screening a library, by performing mutation analysis, by deuterium exchange analysis, by peptide binding assays, and/or by x-ray crystallographic studies. In some embodiments, the validation uses libraries and/or peptides derived from the target protein to assess the importance of specific amino acids in specific positions for binding. In some embodiments, the library comprises a library of mutations to a subset or all of the amino acid residues of the target protein. In some embodiments, the library is a yeast display library. In some embodiments, the library is a phage display library.

In some embodiments, to identify the residues to be edited to reduce or abrogate binding of the antibodies to a target intracellular protein, it is desirable to start with a solved crystal structure of the intracellular protein so that the key contact residue(s) can be identified and substituted for residue(s) that will destroy antigen binding but not hinder its cellular function. This solved structure can be the solved crystal structure of the intracellular protein alone, or that of the intracellular protein bound to the antibody or the antibody fragment.

However, in many cases, a good molecular model could provide the necessary information. In cases where the molecular model is not sufficient, e.g., if the appropriate structural templates do not exist in structural databases, amino acid swapping experiments can provide information on which residues can be targeted for mutation. Alanine scanning mutagenesis (mutating each residue sequentially to Ala) through those regions could identify the key residue(s) involved in antigen-binding (Cunningham B C and Wells J A, Proc Natl Acad Sci USA. 1991 Apr. 15; 88(8): 3407-3411). If changing a single residue to Ala reduced but did not destroy binding, that position could be targeted for more drastic mutations (for example, a substitution that created in a charge difference) to further reduce binding, if desired.

Comparative model building provides a great range of structural templates, and computer programs available ensures that models are becoming increasingly accurate. For example, the solved crystal structure of various proteins has been shown to be very close to the structure predicted by molecular modelling.

As shown herein in Example 3 for GRP78, once the structure of the protein target has been elucidated, a person of ordinary skill in the art can identify the residues that can be modified to decrease antibody binding to an epitope of an organelle protein while leaving sufficient function of that organelle protein for cell viability, as taught in more detail herein. Methods for making polypeptides comprising one or more mutations are well known to one of ordinary skill in the art.

In some embodiments, one or more amino acid residues in or near an identified or known epitope of the target protein are mutated. In some embodiments, 1, 2, 3, 4, 5, 6, 7, 8, 9, 10, 11, 12, 13, 14, 15, 16, 17, 18, 19, 20, 21, 22, 23, 24, or 25 or more amino acid residues of the target protein are mutated to generate a variant target protein. In some embodiments, the one or more mutations create a mutant epitope that destroy one or more (or all) of the target epitopes on the target protein. In some embodiments, the one or more mutations are in a target epitope for a specific antibody. In some embodiments, the one or more mutations are conservative mutations. In some embodiments, the one or more mutations are non-conservative mutations. In some embodiments, the one or more mutations are a mixture of conservative and non-conservative mutations. In some embodiments, the mutation is a substitution, a deletion or an insertion.

In some embodiments, the variant target protein contains at least one amino acid substitution to the amino acid sequence of the epitope of the target protein of the antibody. In some embodiments, the variant target protein contains one or more, two or more, three or more, four or more, five or more, six or more, seven or more, eight or more, nine or more, 10 or more, substitutions to the amino acid sequence of the epitope of the target protein of the antibody. In some embodiments, the variant target protein contains one amino acid substitution to the amino acid sequence of the epitope of the target protein of the antibody. In some embodiments, the variant target protein contains two amino acid substitutions to the amino acid sequence of epitope of the target protein of the antibody. In some embodiments, the variant target protein contains three amino acid substitutions to the amino acid sequence of the epitope of the target protein of the antibody. In some embodiments, the variant target protein contains four amino acid substitutions to the amino acid sequence of the epitope of the target protein of the antibody. In some embodiments, the variant target protein contains five amino acid substitutions to the amino acid sequence of the epitope of the target protein of the antibody.

In particular embodiments of the provided methods, the mutation does not affect the function of the target protein. In some embodiments, the mutation is an inert mutation in which the structure and function of the target protein is retained. In some embodiments, the one or more mutations do not disrupt the function of the polypeptide (e.g., do not disrupt the function of the variant target protein relative to the function of the corresponding unmutated target protein). In some embodiments, the one or more mutations do not disrupt native protein-protein interactions of the protein (e.g., the variant protein retains the ability to form substantially the same protein-protein interactions as the corresponding unmutated target protein). In some embodiments, the one or more mutations do not disrupt the three-dimensional structure of the polypeptide (e.g., the variant target protein retains substantially the same three-dimensional structure as the corresponding unmutated target protein). In some embodiments, the one or more mutations do not disrupt the folding of the protein (e.g., the variant protein retains substantially the same protein folding as the corresponding unmutated target protein). In some embodiments, the one or more mutations do not disrupt the translation of the protein (e.g., the variant protein is translated with the same timing, at the same rate, to the same levels, etc. as the corresponding unmutated target protein). In some embodiments, the one or more mutations do not disrupt the normal cellular localization of the protein (e.g., the variant target protein retains substantially the same cellular localization as the corresponding unmutated target protein). In some embodiments, the one or more mutations do not disrupt any post-translational modifications on the target protein (e.g., the variant target protein retains substantially the same post-translational modification profile as the corresponding unmutated target protein).

In some embodiments, the one or more mutations do not significantly affect the viability of the cell line. In some embodiments, the viability of the modified cell line containing the variant target protein under standard culture and passage conditions is retained compared to the cell line containing the unmutated target protein.

In some embodiments, the cell line is a mammalian cell line. In some embodiments, the one or more mutations do not significantly affect the viability of the mammalian cell line. In some embodiments, the viability of the modified mammalian cell line containing the variant target protein under standard culture and passage conditions is retained compared to the mammalian cell line containing the unmutated target protein.

In some embodiments, the one or more mutations reduce or abrogate binding of the antibody to the target protein. In some embodiments, binding and/or reactivity of one or more antibodies to a variant target protein (e.g., containing a mutant epitope) can be assessed. Methods of assessing binding to a polypeptide are known in the art, and may include, without limitation, performing an ELISA assay, performing western blot analysis, performing a radioimmunoassay (RIA), performing Surface Plasmon Resonance (SPR), performing thermopheresis, performing a competition assay, and performing isothermal titration calorimetry. In some embodiments, binding and/or reactivity to the variant target protein comprising the one or more substitutions is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65, at least about 70%, at least about 75%, at least about 80%, at least about 85%, at least about 90%, at least about 95%, or at least about 99% relative to binding and/or reactivity to the target protein lacking the one or more substitutions. In some embodiments, binding and/or reactivity to the variant target protein comprising the one or more substitutions is reduced by at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, at least about 6 fold, at least about 6.5 fold, at least about 7 fold, at least about 7.5 fold, at least about 8 fold, at least about 8.5 fold, at least about 9 fold, at least about 9.5 fold, at least about 10 fold, at least about 100 fold, or at least about 1000 fold relative to binding and/or reactivity to the target protein lacking the one or more substitutions. In some embodiments, binding and/or reactivity to the variant target protein is eliminated (e.g., binding and/or reactivity is undetectable by the binding assay, e.g., relative to a negative control such as an isotype control).

In some embodiments, the variant target protein that is reduced or abrogated in binding to an antibody exhibits a reduction in affinity of the antibody for the variant target protein. In some embodiments, the affinity of the antibody for the variant target protein is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65, at least about 70%, at least about 75%, at least about 80%, at least about 85%, at least about 90%, at least about 95%, or at least about 99% relative to the affinity of the antibody for the target protein lacking the one or more mutations. In some embodiments, the affinity of the antibody for the variant target protein is reduced by at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, at least about 6 fold, at least about 6.5 fold, at least about 7 fold, at least about 7.5 fold, at least about 8 fold, at least about 8.5 fold, at least about 9 fold, at least about 9.5 fold, at least about 10 fold, at least about 100 fold, or at least about 1000 fold relative to the affinity of the antibody for the target protein lacking the one or more mutations. In some embodiments, the affinity of the antibody for the variant target protein is eliminated (e.g., binding of the antibody to the variant target protein is undetectable).

Methods for measuring antibody affinity for a polypeptide are known in the art, and may include, without limitation, performing an ELISA assay, performing a radioimmunoassay (RIA), performing Surface Plasmon Resonance (SPR), performing thermopheresis, performing a competition assay, and performing isothermal titration calorimetry.

Producing Modified Cell Lines Carrying Variant Target Proteins with Mutant Epitope

In some embodiments, a modified cell line is generated by mutating the epitope of the target protein in the mammalian cell line, such as to reduce or abrogate the binding of the antibody to the epitope on the target protein. In some embodiments, the cell line is a mammalian cell line.

Various mammalian cell culture systems can be employed to produce a cell line that is modified. Examples of suitable host cells include cell lines include, but are not limited to, Chinese hamster ovary (CHO) cells, Baby hamster kidney cells, NSO myeloma cells, monkey kidney COS cells, monkey kidney fibroblast CV-I cells, human embryonic kidney 293 (HEK293) cells, human breast cancer SKBR3 cells, human leukemia Jurkat T cells, dog kidney MDCK cells, human cervical cancer HeLa cells. In some embodiments, the mammalian cell line is a CHO cell line. Examples of a CHO cell line is DHFR CHO cells which are auxotrophic for glycine, thymidine and hypoxanthine. Other CHO cell lines, such as CHO-kl, CHO-S, GS-CHO, with same genomic background are also suitable for recombinant protein expression.

Methods of introducing nucleic acid mutations to a gene of interest are well known in the art (see for example Menke D. Genesis (2013) 51: 618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties) and include targeted homologous recombination, site specific recombinases, PB transposases and genome editing by engineered nucleases. Agents for introducing nucleic acid alterations to a gene of interest can be designed publicly available sources or obtained commercially from Transposagen, Addgene and Sangamo Biosciences.

Exemplary methods used to introduce nucleic acid mutations to a target protein include genome editing using endonucleases, meganucleases, zinc-finger nucleases and transcriptional activator-like effector nucleases (TALENs).

In some embodiments, methods to introduce nucleic acid mutations to the target proteins involves genome editing using engineered endonucleases. In some embodiments, this approach involves a reverse genetics method using artificially engineered nucleases to cut and create specific double-stranded breaks at a desired location(s) in the genome, which are then repaired by cellular endogenous processes such as, homology directed repair (HDR) and non-homologous end-joining (NHEJ). NHEJ directly joins the DNA ends in a double-stranded break, while HDR utilizes a homologous sequence as a donor template for regenerating the missing DNA sequence at the break point. In order to introduce specific nucleotide modifications (e.g., mutations, such as amino acid substitutions) to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and RNA-guided nucleases (RGNs) such as Type II and Type V RGNs.

In certain aspects, including those disclosed herein, the genome editing of the illustrative embodiments utilize clustered regularly interspaced short palindromic repeats (CRISPR) techniques, in which RGNs are used to edit specific target regions in an organism's genome. The canonical CRIPSR systems for genome editing contains distinct components: a guide RNA (“gRNA”), a target region homologous to the gene of interest that contains the intended edit(s), generally referred to as a “homology arm” or “donor template”, and an RGN, e.g., Cas9, Cpf1 or MAD7. By delivering the RGN, synthetic gRNA and donor template into a cell, the cell's genome can be cut at a desired location, allowing edits to the target region of the genome. The guide RNA helps the RGN proteins recognize and cut the DNA of the target genome region based on a recognized protospacer adjacent motif (PAM). The binding of the gRNA localizes the RGN to the genomic target sequence so that the RGN can cut both strands of the DNA, causing a double-strand break. The double-stranded breaks produced by the RGN can undergo homologous directed repair or NHEJ.

Various RGN systems can be for the cell systems and methods disclosed herein. Such systems include, but are not limited to, Type II systems (such as those using RGNs such as Cas9) and Type V systems, such as those using RGNs such as MAD7 (e.g., Wierson W A et al., CRISPR J. 2(6): 417-433 (2019)) or Cpf1 (e.g., Zetsche et al., Cell. 2015 Oct. 22; 163(3): 759-771; Gao L et al., Nat Biotechnol. 35(8): 789-792(2017)); RGN systems using Class I CRISPR systems, such as Cas3 (e.g., Morisaka H. et al., Nat Commun, 2019 December 6; 10(1):5302) or Cas10d, a functional nuclease in the type I-D system (e.g., Osakabe et al., Nucleic Acids Res. 2021 Jun. 21; 49(11): 6347-6363).

In some embodiments, the recombinant nuclease is a Cas9. In some embodiments, the Cas9 is from Streptococcus pyogenes (SpCas9). In some embodiments, the Cas9 is from Staphylococcus aureus (SaCas9). In some embodiments, the Cas9 is from Neisseria meningitidis (NmeCas9). In some embodiments, the Cas9 is from Campylobacter jejuni (CjCas9). In some embodiments, the Cas9 is from Streptococcus thermophilis (StCas9).

In some embodiments, the recombinant nuclease is a Cpf1. In some embodiments, the recombinant nuclease is a modified Cpf1, such as the Alt-R Cas12a system (Integrated DNA Technologies, Coralville, Iowa). In some embodiments, the recombinant nuclease is MAD7.

In some embodiments, the RGN comprises one or more mutations such that the RGN is converted into a nickase that lacks the ability to cleave both strands of a double stranded DNA molecule. In some embodiments, the RGN comprises one or more mutations such that the RGN is converted into a nickase that is able to cleave only one strand of a double stranded DNA molecule. For example, Cas9, which is normally capable of inducing a double strand break, can be converted into a Cas9 nickase, which is capable of inducing a single strand break, by mutating one of two Cas9 catalytic domains: the RuvC domain, which comprises the RuvC I, RuvC II, and RuvC III motifs, or the NHN domain. In some embodiments, the Type II RGN comprises one or more mutations in the RuvC catalytic domain or the HNH catalytic domain. In some embodiments, the recombinant nuclease is a recombinant nuclease that has been modified to have nickase activity. In some embodiments, the recombinant nuclease cleaves the strand to which the guide RNA hybridizes but does not cleave the strand that is complementary to the strand to which the guide RNA hybridizes. In some embodiments, the recombinant nuclease does not cleave the strand to which the guide RNA hybridizes but does cleave the strand that is complementary to the strand to which the guide RNA hybridizes.

In some embodiments, the recombinant nuclease used for editing is a fusion protein that contains both nuclease activity and another enzymatic activity, e.g., reverse transcriptase activity, transposase activity, recombinase activity or other activity that enhances the editing rate in the cells. See, e.g., Newby G A, Liu D R. Mol Ther. 2021 Nov. 3; 29(11):3107-3124. In some embodiments, the genome editing that introduces the one or more desired edits to a target region is base editing, which does not require double-stranded breaks or donor DNA. Anzalone A V, et al., Nature. 2019 December; 576(7785):149-157. doi: 10.1038/s41586-019-1711-4. Epub 2019 October 21. PMID: 31634902.

In certain aspects, the RGN-directed genome editing both introduces one or more desired edits to a target region and removes the proto-spacer motif (PAM) region from the target region, thus precluding any additional editing of the genome at that target region. In this aspect, for example, cells having the desired edit can be selected using an RGN complexed with a synthetic gRNA complementary to the target region. Cells that did not undergo the first editing event will be cut, and thus will not continue to be viable under appropriate selection criteria. The cells containing the desired mutation will not be cut, as they will no longer contain the necessary PAM site.

There are a number of publicly available tools available to help choose and/or design target sequences as well as lists of bioinformatically determined unique gRNAs for targeting specific genome regions in different species using different RGNs, such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder; the Alt-R HDR Design Tool & Templates provided by Integrated DNA Technologies (Coralville, Iowa) and the MAD7 for Gene Editing Design Tool from Horizon (Cambridge, England).

In some embodiments, the RGN and gRNA are introduced into the cell as a ribonucleoprotein (RNP) complex. RNP complexes comprise a protein, such as a recombinant nuclease or a protein comprising a nuclease activity, and a guide RNA. In some embodiments, the recombinant nuclease is provided as a protein, and the guide RNA is provided as a transcribed or synthesized RNA. In some embodiments, the guide RNA forms an RNP complex with the RGN under suitable conditions prior to delivery to the cells.

Although the examples described herein utilize RGN-directed editing systems, it will be apparent to one skilled in the art upon reading the present disclosure that various editing mechanisms can be used to create the cells, systems and methods of manufacture disclosed. Multiple different nuclease-based systems exist for providing edits into an organism's genome, and each can be used in either single editing systems, sequential editing systems (e.g., using different nuclease-directed systems sequentially to provide two or more genome edits in a cell) and/or recursive editing systems, (e.g., utilizing a single nuclease-directed system to introduce two or more genome edits in a cell). Thus, a person of skill in the art would recognize upon reading the present disclosure that various enzyme-directed editing systems are useful for the disclosed embodiments.

For example, in certain embodiments the genomic alterations described herein can be introduced using zinc-finger nuclease genome editing. Zinc-finger nucleases (ZFNs) are artificial restriction enzymes generated by fusing a zinc finger DNA-binding domain to a DNA-cleavage domain. Zinc finger domains can be engineered to target-specific regions in an organism's genome. (Urnov et al., Nature Reviews Genetics, 11:636-646 (2010); International Patent Application Publication WO 2003/087341 A2 to Carroll et al. filed Jan. 22, 2003). Using the endogenous DNA repair machinery of an organism, ZFNs can be used to precisely alter a target region of the genome. ZFNs can be used to produce double-strand breaks (“DSBs”) in the DNA in an allele, which will, in the absence of a homologous template, be repaired by non-homologous end-joining (NHEJ). NHEJ repairs DSBs by joining the two ends together and usually produces no additional mutations, provided that the cut is clean and uncomplicated. (Dural et al., Nucleic Acids Res., 33(18):5978-90 (2005)). This repair mechanism can be used to induce edits in the genome via indels or chromosomal rearrangement.

In selected embodiments, the genomic alterations described herein can be introduced using transcription activator-like effector nuclease editing. Transcription activator-like effector nucleases (TALENs) are restriction enzymes that can be engineered to cut specific sequences of DNA. They are made by fusing a TAL effector DNA-binding domain to a DNA cleavage domain. TALENs can be engineered to bind to practically any desired DNA sequence, so when combined with a nuclease, DNA can be cut at specific locations. (See, e.g., Miller, et al., Nature Biotechnology, 29 (2): 143-8 (2011); Boch, Nature Biotechnology, 29(2): 135-6 (2011); International Patent Application Publication WO 2010/079430 A1 to Bonas et al., filed Jan. 12, 2010; International Patent Application Publication WO 2011/072246 A2 to Voytas et al., filed Dec. 10, 2010).

Like ZFNs, TALENs can edit genomes by inducing DSBs. The TALEN-created site-specific DSBs at target regions are repaired through NHEJ or HDR, resulting in targeted genome edits. TALENs can be used to introduce indels, rearrangements, or to introduce DNA into a genome through NHEJ in the presence of exogenous double-stranded DNA fragments.

ZFNs and TALENs restriction endonuclease technology utilizes a non-specific DNA cutting enzyme which is linked to a specific DNA binding domain (either a series of zinc finger domains or TALE repeats, respectively). Typically, a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fok1. Additionally, Fok1 has the advantage of requiring dimerization to have nuclease activity and this means the specificity increases dramatically as each nuclease partner recognizes a unique DNA sequence. To enhance this effect, Fok1 nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the Fok1 domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g., Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www.talendesign.org). TALEN can also be designed and obtained commercially from e.g., Sangamo Biosciences™ (Richmond, Calif).

In selected embodiments, the genomic alterations described herein can be introduced using meganuclease-directed editing. Meganucleases were identified in the 1990s, and subsequent work has shown that they are particularly promising tools for genome editing, as they are able to efficiently induce homologous recombination, generate mutations in coding or non-coding regions of the genome, and alter reading frames of the coding regions of genomes. (See, e.g., Epinat, et al., Nucleic Acids Research, 31(11): 2952-2962; and U.S. Pat. No. 8,921,332 to Choulika et al., issued Dec. 30, 2014.) The high specificity of meganucleases gives them a high degree of precision and much lower cell toxicity than other naturally occurring restriction enzymes.

Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8, 163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

The introduction of editing systems can be carried out by any suitable delivery means. For example, delivery methods and/or vehicles for introduction or transfer to cells can use viral delivery, e.g., lentiviral, vectors, delivery vectors, or any of the known methods and/or vehicles for delivering such agent(s). In some embodiments, the one or more protein(s) and/or nucleic acids needed for gene editing are introduced or delivered into the cell using electroporation or other physical delivery method, such as microinjection, particle gun, calcium phosphate transfection, or cell compression or squeezing (e.g., as described in Lee, et al, 2012, Nano Lett 12: 6322-27).

In some embodiments, the methods provided herein include introducing or delivering the recombinant nuclease, and in some cases with a gRNA, and introducing or delivering the donor template simultaneously. In some embodiments, the recombinant nuclease, a gRNA, and the donor template are introduced or delivered simultaneously. In some embodiments, the recombinant nuclease, and in some cases with a gRNA, and donor template are introduced or delivered sequentially. In some embodiments, the recombinant nuclease and gRNA are introduced together and are introduced or delivered prior to the introduction or delivery of the donor template.

In other aspects, the genome editing of the illustrative embodiments can utilize homologous recombination methods, including the cre-lox technique and the FRET technique. Site-specific homologous recombination differs from general homologous recombination in that short specific DNA sequences, which are required for the recombinase recognition, are the only sites at which recombination occurs. Site-specific recombination requires specialized recombinases to recognize the sites and catalyze the recombination at these sites. A number of bacteriophage- and yeast-derived site-specific recombination systems, each comprising a recombinase and specific cognate sites, have been shown to work in eukaryotic cells for the purpose of DNA integration and are therefore applicable for use in the present invention, and these include the bacteriophage P1 Cre/lox, yeast FLP-FRT system, and the Dre system of the tyrosine family of site-specific recombinases. Such systems and methods of use are described, for example, in U.S. Pat. Nos. 7,422,889; 7,112,715; 6,956,146; 6,774,279; 5,677,177; 5,885,836; 5,654,182; and 4,959,317. Other systems of the tyrosine family (such as bacteriophage lambda Int integrase, HK2022 integrase), and systems belonging to the separate serine family of recombinases (such as bacteriophage phiC31, R4Tp901 integrases) also work in editing mammalian cells using their respective recombination sites, and are also applicable for use in the present invention. Exemplary methodologies for homologous recombination are described in U.S. Pat. Nos. 6,689,610; 6,204,061; 5,631,153; 5,627,059; 5,487,992; and 5,464,764.

In some embodiments, the Cre/lox system is used as a site-specific recombinase. In some embodiments, the FLP-FRT site-specific recombinase system is used. In some embodiments, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

In some embodiments, a transposase system can be used. A transposon is a mobile genetic element comprising a nucleotide sequence which can move around to different positions within the genome of a single cell. In the process the transposon can cause mutations and/or change the amount of a DNA in the genome of the cell. A number of transposon systems that are able to also transpose in cells e.g., vertebrates have been isolated or designed, such as Sleeping Beauty (Izsvik and Ivics Molecular Therapy (2004) 9, 147-156), piggyBac (Wilson et al. Molecular Therapy (2007) 15, 139-145), Tol2 (Kawakami et al. PNAS (2000) 97 (21): 11403-11408) or Frog Prince (Miskey et al. Nucleic Acids Res. Dec. 1, 2003 31(23): 6873-6881). Typically, the transposase system offers an alternative means for the removal of selection cassettes after homologous recombination similar to the use Cre/Lox or Flp/FRT. Thus, as an example, the piggyback (PB) transposase system involves introduction of a targeting vector with 3′ and 5′ homology arms containing the mutation of interest, two PB terminal repeat sequences at the site of an endogenous TTAA sequence and a selection cassette placed between PB terminal repeat sequences. Positive selection is applied and homologous recombinants that contain targeted mutation are identified. Transient expression of PBase removes in conjunction with negative selection results in the excision of the selection cassette and selects for cells where the cassette has been lost. The final targeted allele contains the introduced mutation with no exogenous sequences. In some embodiments, for the introduction of sequence alterations by transposase systems like pB, there must be a native TTAA site in relatively close proximity to the location where a particular mutation is to be inserted.

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g., chromatography, electrophoretic methods, immunodetection assays such as ELISA and western blot analysis and immunohistochemistry.

In some embodiments, positive and/or negative selection markers can be designed, such as by using a knock-in/knock-out construct, for efficiently selecting transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations and/or elimination of a marker sequence (e.g., positive marker). Non-limiting examples of such negative markers include the herpes simplex-thymidine kinase (HSV-TK) which converts ganciclovir (GCV) into a cytotoxic nucleoside analog, hypoxanthine phosphoribosyltransferase (HPRT) and adenine phosphoribosytransferase (ARPT).

In some embodiments, the variant target proteins, or polynucleotides encoding the same, are sequenced to identify or confirm their one or more substitutions. Methods of sequencing polynucleotides and/or polypeptides are known in the art, including use of sequencing systems from Illumina (San Diego, CA), Pacific Biosciences (Mountain View, CA), Oxford Nanopore Technologies (Oxford, UK), and Element Biosciences (San Diego, CA).

### Expression and Culturing of Modified Cell Lines

In some embodiments, there is provided the use of a modified cell line, such as a mammalian cell line, according to the provided embodiments to produce an antibody by transfection of the cell line with antibody-producing genes. In some embodiments, a nucleic acid encoding the antibody is introduced into the modified mammalian cell line for expressing the antibody in the cell. Methods for introducing a nucleic acid into a cell are well-known to a skilled artisan and includes a variety of art-recognized protocols for the introducing foreign DNA into host cells (see Kaufman, RJ. Meth. Enzymology 185:537 (1988)). Selection of a transformation or transfection protocol will depend upon the host cell and the nature of the transgene and protein product. Commonly used method for introducing exogenous DNA into host cells include calcium phosphate precipitation or DEAE-dextran-medicated transfection. Alternatively, electroporation can be used to introduce DNA directed into the cytoplasm of a host cell, or a reagent (e.g., Lipofectin® Reagent or Lipofectamine® Reagent, Gibco BRL, Gaithersburg, MD) capable of forming lipid-nucleic acid complexes or liposomes which facilitates uptake of nucleic acid into host cells when the complex is applied to cultured cells can be used.

In some embodiments, nucleic acid sequences encoding the antibody can be assembled in, or inserted into, an expression vector. In some embodiments, the modified mammalian cell line is transfected singly with a nucleic acid encoding the heavy (H) chain and light (L) chain of the antibody, or are co-transfected with a nucleic acid encoding the H and L chain. For instance, in one embodiment, nucleic acids encoding the antibody or antigen-binding fragments thereof are assembled in separate expression vectors that are then used to cotransfect the modified cell line. Alternatively, nucleic acid encoding the H and L chains can be assembled on the same expression vector. The modified mammalian cells are cultured under conditions that permit expression of the incorporated genes and the expressed immunoglobulin chains or intact antibodies or fragments are recovered from the culture.

For long-term, high-yield production of recombinant antibodies, stable expression may be used. For example, cell lines, which stably express the antibody molecule may be used for the methods of the disclosure. Rather than using expression vectors which contain viral origins of replication, host cells can be transformed with immunoglobulin expression cassettes and a selectable marker. Following the introduction of the foreign DNA, engineered cells may be allowed to grow, such as for 1-2 days, in enriched media, and then are switched to a selective media. The selectable marker in the recombinant plasmid confers resistance to the selection and allows cells to stably integrate the plasmid into a chromosome and grow to form foci which in turn can be cloned and expanded into cell lines.

An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. The following vectors are provided by way of example and should not be construed in anyway as limiting: Bacterial: pBs, phagescript, PsiX174, pBluescript SK, pBs KS, pNH8a, pNH16a, pNH18a, pNH46a; pTrc99A, pKK223-3, pKK233-3, pDR540, and pRIT5. Eukaryotic: pWLneo, pSV2cat, pOG44, PXR1, pSG, pSVK3, pBPV, pMSG and pSVL.

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g., viral vectors based on vaccinia virus; poliovirus; adenovirus (see, e.g., Li et al., Invest. Opthalmol. Vis. Sci. (1994) 35: 2543-2549; Borras et al., Gene Ther. (1999) 6: 515-524; Li and Davidson, Proc. Natl. Acad. Sci. USA (1995) 92: 7700-7704; Sakamoto et al., H. Gene Ther. (1999) 5: 1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9: 81-86, Flannery et al., Proc. Natl. Acad. Sci. USA (1997) 94: 6916-6921; Bennett et al., Invest. Opthalmol. Vis. Sci. (1997) 38: 2857-2863; Jomary et al., Gene Ther. (1997) 4:683 690, Rolling et al., Hum. Gene Ther. (1999) 10: 641-648; Ali et al., Hum. Mol. Genet. (1996) 5: 591-594; Srivastava in WO 93/09239, Samulski et al., J. Vir. (1989) 63: 3822-3828; Mendelson et al., Virol. (1988) 166: 154-165; and Flotte et al., Proc. Natl. Acad. Sci. USA (1993) 90: 10613-10617); SV40; herpes simplex virus; human immunodeficiency virus (see, e.g., Miyoshi et al., Proc. Natl. Acad. Sci. USA (1997) 94: 10319-23; Takahashi et al., J. Virol. (1999) 73: 7812-7816); or a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus).

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In order to assess the expression of a polypeptide (e.g., an antibody) or portions thereof, the expression vector to be introduced into a cell may also contain either a selectable marker gene or a reporter gene, or both, to facilitate identification and selection of expressing cells from the population of cells sought to be transfected. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, without limitation, antibiotic-resistance genes.

In some embodiments, the expression vector encoding the antibody includes a selectable marker to further amplify expression of the expressed antibody. In some embodiments, a method of amplifying the gene of interest is also desirable and typically involves the use of a selection gene which confers a selectable phenotype. Generally speaking, a “selection gene” is a gene that confers a phenotype on cells that express the gene as a detectable protein. A commonly used example of selection genes include but are not limited to, antibiotic resistance genes. For example, useful dominant selectable markers include microbially derived antibiotic resistance genes, which confer resistance to neomycin, kanamycin or hygromycin when the drug (or selection agent) is added exogenously to the cell culture.

In some embodiments, host mammalian cells provide posttranslational modifications to immunoglobulin protein molecules including leader peptide removal, folding and assembly of heavy and light chains, glycosylation of the antibody molecules, and secretion of functional antibody protein.

Once an antibody has been produced, it may be purified by any method known in the art for purification of an immunoglobulin molecule, for example, by chromatography (ex. ion exchange, affinity, particularly affinity for the specific antigen after Protein A, and sizing column chromatography), centrifugation, differential solubility, or by any other standard technique for the purification of proteins. In many embodiments, antibodies are secreted from the cell into culture medium and harvested from the culture medium.

### Modified Mammalian Cells for ANTI-GRP78 Antibody Production

In some embodiments, the provided methods and cell systems can be used to produce antibodies directed to GRP78, also called anti-GRP78 antibodies. The antibodies may include full-length antibodies or antigen-binding fragments thereof. GRP78, also known as heat shock protein-5 (Bspa5/BiP), is part of an evolutionarily conserved, ER-linked stress response mechanism that provides cellular survival signals during environmental and physiologic duress. As a molecular component of the ER chaperoning network, GRP78 is classically involved with the processing of unfolded proteins, however newer insights also place the protein at the cell surface with the potential to influence signal transduction (Lee, 2014, Nature Rev Cancer 1.4(4); 263-276). Retrospective IHC studies have demonstrated that GRP78 expression positively correlates with poor survival in advanced breast cancer (Lee et al., 2006, Cancer Research 66(16): 7849-7853) and recurrence in prostate cancer patients (Daneshmand et al., 2007, Human Pathology 38(10): 1547-1552). Upregulation of GRP78 promotes survival and chemoresistance in both proliferating and dormant breast cancer cells. Synthetic peptides composed of GRP78-binding motifs coupled to a cell death-inducing peptide to promote apoptosis in cancer cells, demonstrating its in vivo accessibility (Arap et al., 2004, Cancer Cell 6(3): 275-284).

In some embodiments, the antibody comprises an antibody or an antigen-binding fragment thereof. In some embodiments, the antigen-binding fragment is selected from the group consisting of a Fab, a single-chain variable fragment (scFv), a single-domain antibody, and a nanobody. In further embodiments, the antibody is a full-length antibody. In some embodiments, the anti-GRP78 antibody is a humanized antibody. In some embodiments, the anti-GRP78 antibody is a human antibody.

In some embodiments, the antibody is an antibody as described in PCT published App. No. WO2018057703. In some embodiments, the anti-GRP78 is the antibody designated as B4. In some embodiments, the anti-GRP78 antibody is the antibody designated D1. In some embodiments, the anti-GRP78 antibody is the antibody designated F6. In any of such embodiments, the antibody is a full-length antibody of any of the foregoing or is an antigen-binding fragment thereof. In some embodiments, the anti-GRP78 antibody is a full-length antibody.

In some embodiments, the anti-GRP78 antibody includes a variable heavy (VH) chain and a variable light (VL) chain. In some embodiments, the VH chain comprises the VH CDR1 set forth in SEQ ID NO: 1, the VH CDR2 set forth in SEQ ID NO: 2, the VH CDR3 set forth in SEQ ID NO: 3; and the VL chain comprises the VL CDR1 set forth in SEQ ID NO: 4, the VL CDR2 set forth in SEQ ID NO: 5, and the VL CDR3 set forth in SEQ ID NO: 6. In some embodiments, the VH chain has a sequence of amino acids that has at least 85%, at least 90% or at least 95% sequence identity to the sequence set forth in SEQ ID NO: 19 and the VL chain has at least 85%, at least 90% or at least 95% sequence identity to the sequence set forth in SEQ ID NO: 20. In some embodiments, the VH chain is set forth in SEQ ID NO: 19 and the VL chain is set forth in SEQ ID NO:20.

In some embodiments, the VH chain comprises the VH CDR1 set forth in SEQ ID NO: 7, the VH CDR2 set forth in SEQ ID NO: 8, the VH CDR3 set forth in SEQ ID NO: 9; and the VL chain comprises the VL CDR1 set forth in SEQ ID NO: 10, the VL CDR2 set forth in SEQ ID NO: 11, and the VL CDR3 set forth in SEQ ID NO: 12. In some embodiments, the VH chain has a sequence of amino acids that has at least 85%, at least 90% or at least 95% sequence identity to the sequence set forth in SEQ ID NO: 21 and the VL chain has at least 85%, at least 90% or at least 95% sequence identity to the sequence set forth in SEQ ID NO: 22. In some embodiments, the VH chain is set forth in SEQ ID NO: 21 and the VL chain is set forth in SEQ ID NO:22.

In some embodiments, the VH chain comprises the VH CDR1 set forth in SEQ ID NO: 13, the VH CDR2 set forth in SEQ ID NO: 14, the VH CDR3 set forth in SEQ ID NO: 15; and the VL chain comprises the VL CDR1 set forth in SEQ ID NO: 16, the VL CDR2 set forth in SEQ ID NO: 17, and the VL CDR3 set forth in SEQ ID NO: 18. In some embodiments, the VH chain has a sequence of amino acids that has at least 85%, at least 90% or at least 95% sequence identity to the sequence set forth in SEQ ID NO: 23 and the VL chain has at least 85%, at least 90% or at least 95% sequence identity to the sequence set forth in SEQ ID NO: 24. In some embodiments, the VH chain is set forth in SEQ ID NO: 23 and the VL chain is set forth in SEQ ID NO:24.

In some of any embodiments, the anti-GRP78 antibody binds human GRP78. In some embodiments the human GRP78 protein comprises the amino acid set forth in SEQ ID NO: 25.

In some embodiments, the anti-GPR78 antibody binds to a target epitope of GRP78 that includes one or more amino acid residues K113, R261, H265, K268, K271, K272, R279, E329 or D333, with reference to the residue numbering of human GRP78 set forth in SEQ ID NO:25.

In some embodiments, the anti-GPR78 antibody binds to a target epitope of GRP78 that includes one or more amino acid residues from R261, H265, H329, K271, K272 and D333, with reference to residue numbering of human GRP78 set forth in SEQ ID NO:25. In some embodiments, the target epitope includes amino acid residues R261, H265, H329, K271, K272 and D333, with reference to the residue numbering of human GRP78 set forth in SEQ ID NO:25.

In some embodiments, the anti-GPR78 antibody binds to a target epitope of GRP78 that includes one or more amino acid residues R261, R279, K113, K268 and K271, with reference to the residue numbering of human GRP78 set forth in SEQ ID NO:25. In some embodiments, the target epitope includes amino acid residues R261, H265, H329, K271, K272 and D333, with reference to the residue numbering of human GRP78 set forth in SEQ ID NO:25.

In some embodiments, the anti-GRP78 antibody is an antibody that binds to the same epitope on human GRP78 as an antibody containing any of the above sequences (i.e., antibodies that have the ability to cross-compete for binding to human GRP78 with any of the anti-GRP78 antibodies as described). In some embodiments, the reference antibody or antibody fragment for cross-competition studies can be one of the antibodies or antibody fragments described herein, as comparisons are made using equivalent molecules (e.g., a Fab to a Fab, full-length antibody to a full-length antibody, etc.). For example, Biacore analysis, ELISA assays or flow cytometry may be used to demonstrate cross-competition with any of the described antibodies. The ability of a test antibody to inhibit the binding of to human GRP78 demonstrates that the test antibody can compete with the reference antibody for binding to human GRP78 and thus is considered to bind to the same epitope of human GRP78.

In some embodiments, the modified mammalian cell line is engineered with a variant GRP78 that has a mutant epitope comprising a mutation of one or more amino acid residue of the target epitope of the antibody or antigen binding fragment. In some embodiments, the mutation is an amino acid substitution of one or more amino acid residue to change the target epitope. In some embodiments, the mutant epitope includes 1, 2, 3, 4, 5, 6, 7, 8 or 9 amino acid substitutions of the target epitope of the target protein.

In some embodiments, the modified mammalian cell line is engineered with a variant GRP78 that has a sequence of amino acids that contains at least one amino acid substitution compared to the sequence of human GRP78 set forth in SEQ ID NO:25. In some embodiments, the modified cell line is engineered with a variant GRP78 that has at least 85% and less than 100% sequence identity to the sequence of GPR78 set forth in SEQ ID NO:25. In some embodiments, the modified cell line is engineered with a variant GRP78 that has at least 90% and less than 100% sequence identity to the sequence of GPR78 set forth in SEQ ID NO:25. In some embodiments, the modified cell line is engineered with a variant GRP78 that has at least 95% and less than 100% sequence identity to the sequence of GPR78 set forth in SEQ ID NO:25. In some embodiments, the modified cell line is engineered with a variant GRP78 that has at last 1, 2, 3, 4, 5, 6, 7, 8 or 9 amino acid substitutions compared to the sequence of human GRP78 set forth in SEQ ID NO:25.

In some embodiments, the mutation of the mutant epitope is an amino acid substitution selected from one of at least one of (i)-(ix) or is a combination thereof: (i) K113Q, K113 S, K113D or K113T; (ii) R261Q, R261S, R261D, R261T or R261A; (iii) H265Q, H256S, H265D or H256T; (iv) K268Q, K268S, K268D or K268T; (v) K271Q, K271S, K271D or K271S; (vi) K272Q, K272S, K271D or K272S; (vii) R279Q, R279S, R279D, R279T or R279A; (viii) E329Q, E329S, E329D, E329T or E329A; or (ix) D333Q, D333S or D333T. In some embodiments, the mutation of the mutant epitope is an amino acid substitution from one of at least one of (i)-(ix) or is a combination thereof: (i) K113Q; (ii) R261Q or R261A; (iii) H265Q; (iv) K268Q; (v) K271Q; (vi) K272Q; (vii) R279Q or R279A; (viii) E329Q or E329A; or (ix) D333Q.

In some embodiments, the variant GRP78 contains the mutation R261Q. In some embodiments, the variant GRP78 contains the mutation H265Q. In some embodiments, the variant GRP78 contains the mutation R279Q. In some embodiments, the variant GRP78 contains the mutation E329Q. In some embodiments, the variant GRP78 contains the mutation R261Q and H265Q. In some embodiments, the variant GRP78 contains the mutation R261Q, H265Q, R279Q and E329Q. In some embodiments, the variant GRP78 contains the mutation R261Q, H265Q, and E329Q. In some embodiments, the variant GRP78 contains the mutation R261Q and R279Q. In some embodiments, the variant GRP78 contains the mutation R261Q, H265Q, K271Q, R279Q and E329Q. In some embodiments, the variant GRP78 contains the mutation R261Q, H265Q, K271Q, K272Q, E329Q and D333Q. In some embodiments, the variant GRP78 contains the mutation K113Q, R261Q, K268Q, K271Q and R279Q. In some embodiments, the variant GRP78 contains the mutation K113Q, R261Q, H265Q, K268Q, K271Q, K272Q, R279Q, E329Q and D333Q. In some embodiments, the variant GRP78 contains the mutation R261A, H265A and E329A; R261A and R279A. In some embodiments, the variant GRP78 contains the mutation R261A, H265A, R279A and E329A.

Provided herein is a modified mammalian cell line that is engineered with a variant GRP78 set forth in SEQ ID NO: 26.

Provided herein is a modified mammalian cell line that is engineered with a variant GRP78 set forth in SEQ ID NO:27.

In some embodiments, the mammalian cell line may include Chinese hamster ovary (CHO) cells, Baby hamster kidney cells, NSO myeloma cells, monkey kidney COS cells, monkey kidney fibroblast CV-I cells, human embryonic kidney 293 (HEK293) cells, human breast cancer SKBR3 cells, Human Jurkat T cells, Dog kidney MDCK cells, and Human cervical cancer HeLa cells. In some embodiments, the mammalian cell line is a CHO cell line. In some embodiments, the mammalian cell line is an HEK293 cell line.

In some embodiments, the anti-GRP78 antibody exhibits reduced or abrogated binding to the variant GRP78 containing the mutant epitope. In some embodiments, binding and/or reactivity of the anti-GRP78 to the variant GPR78 is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65, at least about 70%, at least about 75%, at least about 80%, at least about 85%, at least about 90%, at least about 95%, or at least about 99% relative to binding and/or reactivity to human GRP78 lacking the one or more substitutions, e.g., human GRP78 set forth in SEQ ID NO:25. In some embodiments, binding and/or reactivity of the anti-GRP78 to the variant GPR78 is reduced by at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, at least about 6 fold, at least about 6.5 fold, at least about 7 fold, at least about 7.5 fold, at least about 8 fold, at least about 8.5 fold, at least about 9 fold, at least about 9.5 fold, at least about 10 fold, at least about 100 fold, or at least about 1000 fold relative to binding and/or reactivity to human GRP78 lacking the one or more substitutions, e.g., as set forth in SEQ ID NO:25. Methods for assessing binding may include any as described herein.

In some embodiments, the anti-GRP78 antibody exhibits a reduction in affinity of the for the variant GRP78 relative to human GRP78 lacking the one or more substitutions, e.g., set forth in SEQ ID NO:25. In some embodiments, the affinity of the anti-GRP78 antibody for the variant GRP78 is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65, at least about 70%, at least about 75%, at least about 80%, at least about 85%, at least about 90%, at least about 95%, or at least about 99% relative to the affinity for human GRP78 lacking the one or more amino acid substitutions, e.g., set forth in SEQ ID NO:25. In some embodiments, the affinity of the anti-GRP78 antibody for the variant GRP78 is reduced by at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, at least about 6 fold, at least about 6.5 fold, at least about 7 fold, at least about 7.5 fold, at least about 8 fold, at least about 8.5 fold, at least about 9 fold, at least about 9.5 fold, at least about 10 fold, at least about 100 fold, or at least about 1000 fold relative to the affinity for human GRP78 lacking the one or more amino acid substitutions, e.g., as set forth in SEQ ID NO:25. Methods for measuring affinity may include any as described herein.

In some embodiments, the modified mammalian cell line containing a variant GPR78 can be used to produce an anti-GRP78 antibody in accord with any of the provided methods.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting of” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known. Reference to “about” a value or parameter herein includes (and describes) embodiments that are directed to that value or parameter per se. For example, description referring to “about X” includes description of “X”.

The term “antibody” as used herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, heavy chain variable (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific or trispecific, antibodies, diabodies, triabodies, and tetrabodies, tandem di-scFv, tandem tri-scFv.

Unless otherwise stated, the term “antibody” should be understood to explicitly encompass functional antibody fragments thereof also referred to herein as “antigen-binding fragments.” The term also encompasses intact or full-length antibodies, including antibodies of any class or sub-class, including IgG and sub-classes thereof, IgM, IgE, IgA, and IgD.

The terms “complementarity determining region,” and “CDR,” synonymous with “hypervariable region” or “HVR,” are known to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); Martin et al., “Modeling antibody hypervariable loops: a combined algorithm,” PNAS, 1989, 86(23):9268-9272, (“AbM” numbering scheme); and Ye et al., “IgBLAST: an immunoglobulin variable domain sequence analysis tool,” Nucleic Acids Res. 2013 July; 41(Web Server issue):W34-40, (“IgBLAST numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software. The IgBLAST scheme is based on matching to germline V, D and J genes, and can be determined using National Center for Biotechnology Information (NCBI)'s IgBLAST tool.

Table 1, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26--H32.34 | H26--H35B | H30--H35B |
| (Kabat | | | | |
| Numbering¹) | | | | |
| CDR-H1 | H31--H35 | H26--H32 | H26--H35 | H30--H35 |
| (Chothia | | | | |
| Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Under the Kabat numbering scheme, in some embodiments, the CDR amino acid residues in the heavy chain variable domain (VH) are numbered 26-35 (HCDR1), 50-65 (HCDR2), and 95-105 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-35 (HCDR1). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-105 (HCDR3) in a VH; and amino acid residues 24-34 (LCDR1), 50-56 (LCDR2), and 89-97 (LCDR3) in a VL.

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, specific CDR sequences are specified. Exemplary CDR sequences of provided antibodies are described using various numbering schemes (see e.g., Section II), although it is understood that a provided antibody can include CDRs as described according to any of the other aforementioned numbering schemes or other known numbering schemes.

Likewise, unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM, IgBLAST, IMGT, or Contact method, or other known schemes. In other cases, the particular amino acid sequence of a CDR or FR is given.

Tolerable variations of the CDR sequences will be known to those of skill in the art. For example, in some embodiments the polypeptide comprises a complementarity determining region (HCDR or LCDR) that comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any CDR amino acid sequence.

The term “variable region” or “variable domain” refers to the domain of an antibody heavy or light chain that is involved in binding the antibody to antigen. The variable regions of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

As used herein, the terms “culture,” “culturing,” “grow,” “growing,” “maintain,” “maintaining,” “expand,” “expanding,” etc., when referring to cell culture itself or the process of culturing, can be used interchangeably to mean that a cell is maintained outside the body (e.g., ex vivo) under conditions suitable for survival. Cultured cells are allowed to survive, and culturing can result in cell growth, differentiation, or division.

As used herein, an “epitope” refers to any polypeptide determinant capable of specifically binding to an antibody. In certain embodiments, an epitope is a region of an antigen that is specifically bound by an antibody. In certain embodiments, an epitope may include chemically active surface groupings of molecules such as amino acids, sugar side chains, phosphoryl, or sulfonyl groups. In certain embodiments, an epitope may have specific three-dimensional structural characteristics (e.g., a “conformational” epitope) and/or specific charge characteristics. Epitopes can be formed both from contiguous and/or juxtaposed noncontiguous residues (for example, amino acids) of the target molecule. Epitopes formed from contiguous residues (for example, amino acids) typically are retained on exposure to denaturing solvents whereas epitopes formed by tertiary folding typically are lost on treatment with denaturing solvents. An epitope may include but is not limited to at least 3, at least 5 or 8-10 amino acid residues. In some embodiments, an epitope is less than 20 amino acid residues in length, less than 15 residues or less than 12 residues. Two antibodies may bind the same epitope within an antigen if they exhibit competitive binding for the antigen. An epitope can be identified by various scans as well, for example an alanine or arginine scan can indicate one or more residues that the antigen-binding molecule can interact with.

As used herein, the term “mutant epitope” refers to an epitope that has been genetically engineered to comprise one or more non-naturally occurring mutations that alter the sequence of the epitope recognized by an antibody. In some embodiments, the mutation is one or more amino acid substitution of amino acid residues that make up an epitope recognized by an antibody.

The term “expression” or “expressed” as used herein in reference to a gene refers to the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989, Molecular Cloning: A Laboratory Manual, 18.1-18.88).

The term “gene” can refer to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, guide RNA (e.g., a small guide RNA), or micro-RNA.

An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; heavy chain variable (V_{H}) regions, single-chain antibody molecules such as scFvs and single-domain antibodies comprising only the V_{H} region; and multispecific antibodies formed from antibody fragments. In some embodiments, the antibody is or comprises an antibody fragment comprising a variable heavy chain (V_{H}) and a variable light chain (V_{L}) region. In particular embodiments, the antibodies are single-chain antibody fragments comprising a heavy chain variable (V_{H}) region and/or a light chain variable (V_{L}) region, such as scFvs.

Single-domain antibodies (sdAbs) are antibody fragments comprising all or a portion of the heavy chain variable region or all or a portion of the light chain variable region of an antibody. In certain embodiments, a single-domain antibody is a human single-domain antibody.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are produced by enzyme digestion of a naturally-occurring intact antibody. In some aspects, the antibody fragments are scFvs.

A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. The term includes antigen-binding fragments of human antibodies.

The term “humanized” is used to describe antibodies wherein complementarity determining regions (CDRs) from a mammalian animal, e.g., a mouse, are combined with a human framework region. Often polynucleotides encoding the isolated CDRs will be grafted into polynucleotides encoding a suitable variable region framework (and optionally constant regions) to form polynucleotides encoding complete antibodies (e.g., humanized or fully-human), antibody fragments, and the like. Additionally, “humanized” antibodies may be chimeric, human-like, humanized or fully human, in order to reduce their potential antigenicity, without reducing their affinity for their organelle protein target. Chimeric, human-like and humanized antibodies have generally been described in the art.

Humanized antibodies have variable region framework residues that are substantially from a human therapeutic antibody (termed an acceptor antibody) and complementarity determining regions substantially from a mouse-antibody, (referred to as the donor immunoglobulin). See, Queen et al., Proc. Natl. Acad. Sci. USA 86:10029-10033 (1989), WO 90/07861, U.S. Pat. Nos. 5,693,762, 5,693,761, 5,585,089, 5,530,101, and Winter, U.S. Pat. No. 5,225,539. The constant region(s), if present, are also substantially or entirely from a human immunoglobulin. The human variable domains are usually chosen from human antibodies whose framework sequences exhibit a high degree of sequence identity with the murine variable region domains from which the CDRs were derived. The heavy and light chain variable region framework residues can be derived from the same or different human therapeutic antibody sequences. The human therapeutic antibody sequences can be the sequences of naturally occurring human antibodies or can be consensus sequences of several human antibodies. See Carter et al., WO 92/22653. Certain amino acids from the human variable region framework residues are selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid should usually be substituted by the equivalent framework amino acid from the mouse antibody when it is reasonably expected that the amino acid: i) noncovalently binds antigen directly, ii) is adjacent to a CDR region, iii) otherwise interacts with a CDR region (e.g., is within about 6 A of a CDR region), or iv) participates in the VL-VH interface.

Other candidates for substitution are acceptor human framework amino acids that are unusual for a human antibody at that position. These amino acids can be substituted with amino acids from the equivalent position of the mouse donor antibody or from the equivalent positions of more typical human antibodies. Other candidates for substitution are acceptor human framework amino acids that are unusual for a human antibody at that position. The preferred variable region frameworks of humanized antibodies usually show at least 75%, more preferably 80%, and even more preferably 85% sequence identity to a human variable region framework sequence or consensus of such sequences.

In certain examples, a mouse monoclonal antibody can be used as the basis for producing human therapeutic biologics. In one method, for illustration and not limitation, the heavy chain variable VH region is cloned by RT-PCR using mRNA prepared from hybridoma cells. Consensus primers are employed to VH region leader peptide encompassing the translation initiation codon as the 5′ primer and a g2b constant regions specific 3′ primer. The sequences from multiple, independently-derived clones, can be compared to ensure no changes are introduced during amplification. The sequence of the VH region can also be determined or confirmed by sequencing a VH fragment obtained by 5′ RACE RT-PCR methodology and the 3′ g2b specific primer.

The light chain variable V_{L} region of a mouse monoclonal antibody can be cloned in an analogous manner as the V_{H} region. In one approach, a consensus primer set designed for amplification of murine V_{L} regions is designed to hybridize to the V_{L} region encompassing the translation initiation codon, and a 3′ primer specific for the murine Ck region downstream of the V-J joining region. In a second approach, 5′RACE RT-PCR methodology is employed to clone a V_{L} encoding cDNA. The cloned sequences are then combined with sequences encoding human constant regions.

In one approach, the heavy and light chain variable regions are re-engineered to encode splice donor sequences downstream of the respective VDJ or VJ junctions, and cloned into the mammalian expression vector, such as pCMV-hγ1 for the heavy chain, and pCMV-hκ1 for the light chain. These vectors encode human γ1 and Ck constant regions as exonic fragments downstream of the inserted variable region cassette. Following sequence verification, the heavy chain and light chain expression vectors can be co-transfected into COS cells to produce chimeric antibodies. Conditioned media is collected 48 hours post transfection and assayed by western blot analysis for antibody production or ELISA for antigen binding. The chimeric antibodies are preferably humanized as described above.

The heavy and light chain variable regions of chimeric and/or humanized antibodies can be linked to at least a portion of a human constant region of choice. The choice of constant region may be driven by the desired mechanism of action of the antibody, e.g., whether cellular mediated toxicity is desired. For example, isotopes IgG1 and IgG3 have antibody-dependent complement activity and isotypes IgG2 and IgG4 do not. Light chain constant regions can be lambda or kappa. Antibodies can be expressed as tetramers containing two light and two heavy chains, as separate heavy chains, light chains, as Fab, Fab′ F(ab′)2, and Fv, or as single chain antibodies in which heavy and light chain variable domains are linked through a linker.

The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

As used herein, the term “specifically binds” to a target protein or epitope is a term that is well understood in the art, and methods to determine such specific binding are also well known in the art. A molecule is said to exhibit “specific binding” if it reacts or associates more frequently, more rapidly, with greater duration and/or with greater affinity with a particular target protein than it does with alternative proteins. It is also understood that “specific binding” does not necessarily require (although it can include) exclusive binding. Generally, but not necessarily, reference to binding means preferential binding. “Specificity” refers to the ability of a binding protein to selectively bind an antigen.

As used herein, a “stress protein” refer to a protein that functions in normal cells and may be present at high levels under stressful conditions, such as hypoxia, nutrient deprivation, pH changes, oxidative stress, or other metabolic dysregulation of a cell, such as often occurs in cancer cells. A stress protein includes proteins whose expression is increased when the capacity of a cellular organelle, such as the endoplasmic reticulum (ER) or Golgi apparatus, becomes insufficient. A stress protein of the ER includes a protein associated with the unfolded protein response (UPR) that acts to reduce ER stress and restore homeostasis, a protein associated with endoplasmic-reticulum-associated protein degradation (ERAD), or a protein associated with ER stress-mediated apoptosis. Exemplary ER stress proteins include calreticulin, heat shock proteins, and isomerases. A stress protein of the Golgi apparatus includes proteins involved in post-translational modifications such as glycosylation or that are involved in vesicular transport. Exemplary Golgi stress proteins include, for example, GOLPH3.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

As used herein the terms “polypeptide”, “peptide” and “protein” are used interchangeably herein to refer to polymers of amino acids of any length. The polymer may be linear, cyclic, or branched, it may comprise modified amino acids, and it may be interrupted by non amino acids. The terms also encompass amino acid polymers that have been modified, for example, via sulfation, glycosylation, lipidation, acetylation, phosphorylation, iodination, methylation, oxidation, proteolytic processing, phosphorylation, prenylation, racemization, selenoylation, transfer-RNA mediated addition of amino acids to proteins such as arginylation, ubiquitination, or any other manipulation, such as conjugation with a labeling component. As used herein the term “amino acid” refers to either natural and/or unnatural or synthetic amino acids, including glycine and both the D or L optical isomers, and amino acid analogs.

A “vector” is a nucleic acid molecule, preferably self-replicating, which transfers an inserted nucleic acid molecule into and/or between host cells. An “expression vector” is a polynucleotide sequence which, when introduced into an appropriate host cell, can be transcribed and translated into a polypeptide(s). An “expression system” usually connotes a suitable host cell comprised of an expression vector that can function to yield a desired expression product.

The term “recombinant” as applied to a polynucleotide means that the polynucleotide is the product of various combinations of cloning, restriction and/or ligation steps, and other procedures that result in a construct that is distinct from a polynucleotide found in nature.

As used herein the terms “operably linked” or “operatively linked” are used to refer the DNA sequences which are juxtaposed in a manner such that the components so described are in a relationship permitting them to function in their intended manner. For example, a promoter is operably linked to a coding sequence if it controls the transcription of the sequence; or a ribosome binding site is operably linked to a coding sequence if it is positioned so as to permit translation. DNA for a signal sequence (secretory leader) is operably linked to DNA for a polypeptide if it is expressed as a precursor which participates in the secretion of the polypeptide. Generally, operably linked means contiguous.

In some preferred embodiments, this disclosure provides methods of recombinant production of an antibody, the method comprising expressing a nucleic acid encoding an antibody in a modified cell line, wherein the antibody specifically binds to a target epitope on a target protein that is natively expressed as an intracellular protein in the cell; and wherein the modified cell is engineered to express a variant target protein that comprises a mutation of one or more amino acid residues of the target epitope; and, culturing the modified cell line under conditions so that the antibody is produced. In some preferred embodiments, the further comprises isolating the antibody from the culture supernatant. In some embodiments, the target protein that is natively expressed is an intracellular protein in the secretory pathway of the cell line. In some preferred embodiments, the mutation is an inert mutation that retains the structure and function of the native target protein in the cell. In some preferred embodiments, the mutation reduces or abrogates binding of the antibody to the target epitope on the target protein. In some preferred embodiments, the target protein is a stress protein. In some preferred embodiments, the target protein is expressed on an organelle of the cell line. In some preferred embodiments, the organelle is the endoplasmic reticulum or the Golgi apparatus. In some preferred embodiments, the target protein is an endoplasmic reticulum chaperone. In some preferred embodiments, the endoplasmic reticulum chaperone is a calreticulin, a heat shock protein or an isomerase. In some preferred embodiments, the target protein is glucose-regulated protein 78 (GRP78), HSP47, PDI, calreticulin or GP94. In some preferred embodiments, the target protein is glucose-regulated protein 78 (GRP78). In some preferred embodiments, the target protein is a Golgi complex protein. In some preferred embodiments, the Golgi complex protein is GOLPH2, GOLPH3, GM130, ATP6V1A, ATP6V1E1, ATP6VOA2, TMEM165, GOLGB1, SCYL1BP1, TRAPPC11, TRAPPC2 or TRIP11.

A method of recombinant production of an antibody, the method comprising: expressing a nucleic acid encoding an antibody in a modified mammalian cell line, wherein the antibody specifically binds a target epitope on a target protein that is natively expressed as an intracellular protein in the mammalian cell line and wherein the modified mammalian cell line is engineered to express a variant target protein that comprises a mutation of one or more amino acid residues of the target epitope; and, culturing the modified mammalian cell line under conditions so that the antibody is produced. In some preferred embodiments, the methods further comprise isolating the antibody from the culture supernatant. In some preferred embodiments, the target protein that is natively expressed is an intracellular protein in the secretory pathway of the mammalian cell line. In some preferred embodiments, the mutation is an inert mutation that retains the structure and function of the target protein in the mammalian cell line. In some preferred embodiments, the mutation reduces or abrogates binding of the antibody to the target epitope on the target. In some preferred embodiments, the target protein is a stress protein. In some preferred embodiments, the target protein is expressed on an organelle of the mammalian cell line. In some preferred embodiments, the organelle is the endoplasmic reticulum or the Golgi apparatus. In some preferred embodiments, the target protein is an endoplasmic reticulum chaperone. In some preferred embodiments, the endoplasmic reticulum chaperone is a calreticulin, a heat shock protein or an isomerase. In some preferred embodiments, the target protein is glucose-regulated protein 78 (GRP78), HSP47, PDI, calreticulin or GP94. In some preferred embodiments, the target protein is glucose-regulated protein 78 (GRP78). In some preferred embodiments, the target protein is a Golgi complex protein. In some preferred embodiments, the Golgi complex protein is GOLPH2, GOLPH 3, GM130, ATP6V1A, ATP6V1E1, ATP6VOA2, TMEM165, GOLGB1, SCYL1BP1, TRAPPC11, TRAPPC2 or TRIP11.

In some preferred embodiments, this disclosure provides methods of recombinant production of an antibody targeting an epitope on a target protein found intracellularly in a cell line, the method comprising providing an antibody that binds to an epitope on a target protein that is present intracellularly in a cell line; identifying the epitope of the target protein to which the antibody binds; generating a modified cell line by mutating the epitope of the target protein in the cell line to reduce or abrogate the binding of the antibody to the epitope on the target protein; introducing an expression vector encoding the antibody to the modified cell line; culturing the modified cell line under conditions that allow production of the antibody from the expression vector; and, expressing a nucleic acid encoding an antibody in the modified cell line. In some preferred embodiments, the produced antibody is secreted to the culture supernatant. In some preferred embodiments, the method further comprises isolating the antibody or antigen binding fragment. In some preferred embodiments, the antibody is a humanized antibody. In some preferred embodiments, the antibody is a human antibody. In some preferred embodiments, the target protein that is present intracellularly in the cell line is an intracellular protein in the secretory pathway of the cell line. In some preferred embodiments, the target protein is a stress protein.

In some preferred embodiments, this disclosure provides methods of recombinant production of an antibody targeting an epitope on a target protein found intracellularly in a mammalian cell line, the method comprising: providing an antibody that binds to an epitope on a target protein that is present intracellularly in a mammalian cell line; identifying the epitope of the target protein to which the antibody binds; generating a modified cell line by mutating the epitope of the target protein in the mammalian cell line to reduce or abrogate the binding of the antibody to the epitope on the target protein; introducing an expression vector encoding the antibody to the modified mammalian cell line; culturing the modified mammalian cell line under conditions that allow production of the antibody from the expression vector; and, expressing a nucleic acid encoding an antibody in the modified mammalian cell line. In some preferred embodiments, the produced antibody is present in the culture supernatant. In some preferred embodiments, the methods further comprise isolating the antibody or antigen binding fragment. In some preferred embodiments, the antibody is a humanized antibody. In some preferred embodiments, the antibody is a human antibody. In some preferred embodiments, the target protein that is present intracellularly in the mammalian cell line is an intracellular protein in the secretory pathway of the mammalian cell line. In some preferred embodiments, the target protein is a stress protein.

In some preferred embodiments, this disclosure provides methods of recombinant production of an antibody targeting GRP78, the method comprising: expressing a nucleic acid encoding an antibody in a modified mammalian cell line, wherein the antibody binds to GRP78 and wherein the modified mammalian cell is engineered with a variant GRP78 that has a mutant epitope comprising a mutation of one or more amino acid residues of the target epitope of the antibody or antigen binding fragment; and, culturing the modified mammalian cell line under conditions so that the anti-GRP78 antibody is produced in the culture supernatant. In some preferred embodiments, the methods further comprise isolating the anti-GRP78 antibody from the culture supernatant. In some preferred embodiments, the antibody is a humanized antibody. In some preferred embodiments, the antibody is a human antibody.

In some preferred embodiments, this disclosure provides a modified mammalian cell line comprising a variant protein that is an intracellular protein in the secretory pathway, wherein the variant protein comprises a mutation of a native intracellular protein that is a target protein of an antibody and wherein the mutation is an amino acid substitution(s) of one or more amino acid residues to change a target epitope of the antibody to a mutant epitope. In some embodiments, the mutation is an inert mutation that retains the structure and function of the target protein of the mammalian cell. In some embodiments, the mutation reduces or abrogates binding of the antibody to the variant protein. In some embodiments, the target protein is a stress protein. In some preferred embodiments, this disclosure provides a modified mammalian cell line comprising a variant GRP78 comprising the sequence set forth in SEQ ID NO:28. In some preferred embodiments, this disclosure provides a variant GRP78 comprising the sequence set forth in SEQ ID NO:29. In some preferred embodiments, the mammalian cell line is selected from the group consisting of Chinese hamster ovary (CHO) cells, Baby hamster kidney cells, NSO myeloma cells, monkey kidney COS cells, monkey kidney fibroblast CV-I cells, human embryonic kidney 293 (HEK293) cells, human breast cancer SKBR3 cells, human leukemia Jurkat T cells, dog kidney MDCK cells, and human cervical cancer HeLa cells.

In some preferred embodiments, this disclosure provides methods of recombinant production of an antibody, the method comprising: expressing a nucleic acid encoding an antibody in a modified mammalian cell line, wherein the antibody specifically binds a target epitope on an intracellular signaling protein that is natively expressed in the mammalian cell line and wherein the modified mammalian cell line is engineered to express a variant intracellular signaling protein that comprises a mutation of one or more amino acid residues of the target epitope; and, culturing the modified mammalian cell line under conditions so that the antibody is produced. In preferred embodiments, the methods comprise isolating the antibody from the culture supernatant. In preferred embodiments, the intracellular signaling protein is a phosphatase. In some preferred embodiments, the intracellular signaling protein is a kinase.

Other embodiments are also contemplated herein as would be understood by those of ordinary skill in the art.

### EXAMPLES

The following examples are included for illustrative purposes only and are not intended to limit the scope of the invention.

### Example 1 Production and Purification of Anti-GPRP78 Antibodies in Mammalian Cells

Fully human anti-GRP78 monoclonal antibodies (mAbs) formatted as full-length IgG1 antibodies (Table E1) were individually transfected into Chinese Hamster Ovary (CHO) cells by electroporation in 6-well plates, topped up with fresh media the following day and incubated for 7 days post-transfection. Transfection was carried out using the Neon™ transfection system and Neon™ transfection system reagents (Thermo Fisher Scientific, Waltham, MA) using pre-set settings for CHO cells. Day 7 post-transfection supernatant was loaded onto 1 mL MabSelect PrisimA™ columns to purify the antibody. Antibody concentration and IgG titer in the supernatant was determined using the Octet QK 384 using Protein A biosensors (Molecular Devices, Wokingham, Berkshire, UK), using an IgG1 antibody as standard.

During the culture and at the time of harvest the transfected CHO cells exhibited reduced viability. The supernatant titers for all anti-GRP78 antibodies produced from the CHO cells were low and less than 5 μg/mL (Table E2). To confirm the reduced titer was not specific to CHO cells, the transfections were repeated in Human embryonic kidney 293 (HEK293) cells and antibody was purified from Day 5 supernatant using similar methods, but the titer also remained low (Table E2).

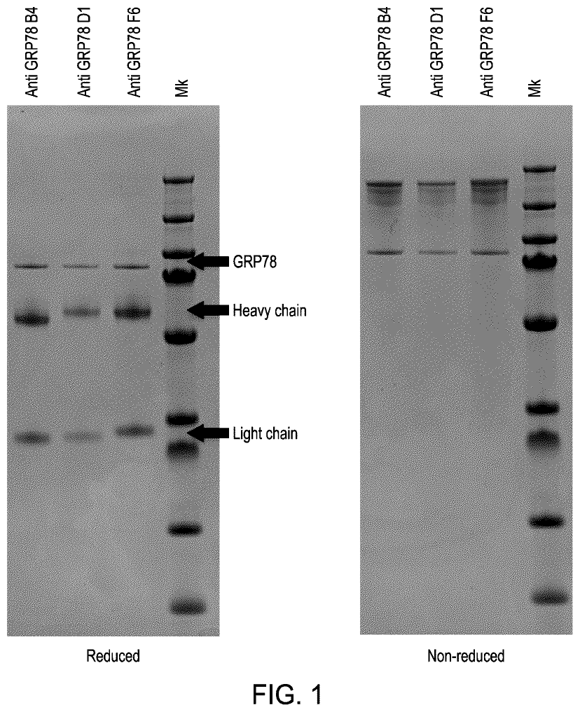

These results are consistent with an observation that production of anti-GRP78 antibody in mammalian cells, such as human cells, is detrimental to the cells and impacts the ability to produce antibody in high yield. It is hypothesized that the antibodies may be binding native GRP78 intracellularly in the endoplasmic reticulum (ER), thereby inhibiting its natural chaperone role in the cell and killing the cells before the antibodies can be secreted into the supernatant for purification. The binding of GRP78 antibodies to the native, ER protein has been demonstrated by experiments showing co-immunoprecipitation of the antibodies with GRP78 as demonstrated by SDS-PAGE analysis of purified sample run under reducing and non-reducing conditions. As shown in FIG. 1, all samples displayed typical SDS-PAGE profile for monomeric IgG, however, there was an extra band between 70-80 kDa consistent with the presence of bound GRP78 antigen.

TABLE E1
Anti-GRP78 Antibodies (disclosed in U.S. Pat. No. 11,254,751)

| | Heavy chain sequences | | | | Light chain sequences | | | |
|---|---|---|---|---|---|---|---|---|
| | (SEQ ID NO) | | | | (SEQ ID NO) | | | |
| | CDR1 | CDR2 | CDR3 | VH | CDR1 | CDR2 | CDR3 | VL |
| B4 | 1 | 2 | 3 | 19 | 4 | 5 | 6 | 20 |
| D1 | 7 | 8 | 9 | 21 | 10 | 11 | 12 | 22 |
| F6 | 13 | 14 | 15 | 23 | 16 | 17 | 18 | 24 |

TABLE E2
Antibody Production in Mammalian Cells

| | Concentration | Total Amount | CHO Day 7 | HEK Day 5 |
|---|---|---|---|---|
| Clone | (mg/mL) | (mg) | Titers (μg/mL) | Titers (μg/mL) |
| B4 | 0.799 | 0.80 | 4.0 | 2.1 |
| D1 | 0.098 | 0.06 | 0.9 | Low |
| F6 | 0.497 | 0.30 | 2.5 | 1.3 |

### Example 2 Epitope Mapping of Anti-GRP78 Antibodies

To characterize the binding properties of the antibodies described in Example 1 for GRP78, epitope mapping analysis was carried out by alanine scan mutagenesis. An alanine scan library of GRP78 was constructed. Each anti-GRP78 antibody was then screened for binding to each individual GRP78 variant, allowing identification of the target protein residues involved in antibody binding.

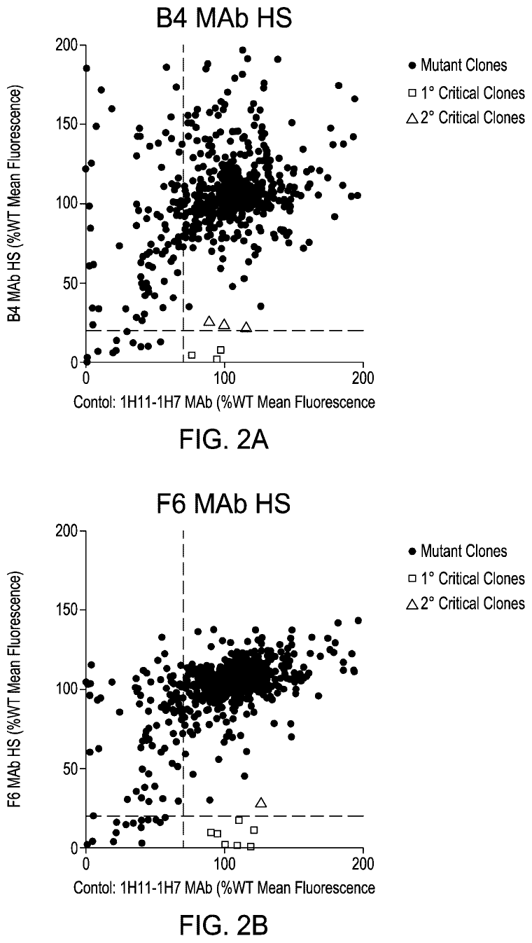

Binding of each test antibody to each GRP78 variant in the alanine scanning library was determined by high-throughput flow cytometry under high stringency conditions (e.g., increased pH, increased salinity, increased temperature and/or increased wash time). Also included in the experiment was a commercially-available monoclonal antibody, 1H11-1H7, which was verified to bind to WT GFR78 on cells and thus determined to be a suitable positive control under these conditions (Thermo Fisher Scientific, Waltham, MA). Antibody binding to each of the GRP78 variant as measured by the fluorescence signal (raw fluorescence data minus the background), was normalized to its binding to the wildtype (WT) GRP78. For each GRP78 variant, such normalized binding to the test antibodies was plotted relative to that to the control antibody. Exemplary results for two anti-GRP78 antibody clones are shown in FIG. 2A and FIG. 2B. GRP78 variants which exhibited >70% binding to control antibody but <20% binding to the test antibody were identified as primary and major binding residues for the test antibody. Other GRP78 variants which did not meet the criteria above but showed decreased binding activity (20%-30%) and close proximity (based on the known 3D structure of the protein) to the above identified primary residues, also were identified (and considered as “other residues involved in antibody binding”).

Table E3 sets forth the residues involved in antibody binding for exemplary anti-GRP78 clones B4 and F6.

TABLE E3
Residues involved in antibody binding to GRP78

| | |
|---|---|
| B4 | Major: R261, H265, E329 |
| | Others: K271, K272, D333 |
| F6 | Major: R261, R279 |
| | Others: K113, K268, K271 |

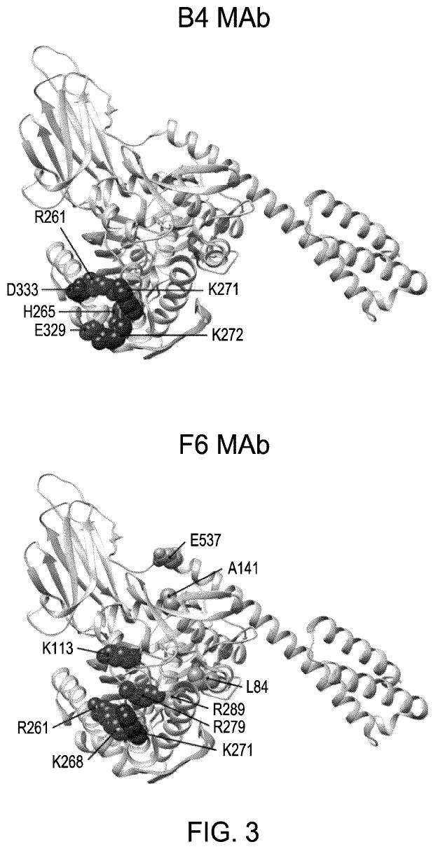

PDB ID 6ASY (Yang et al., Nature Communications. 2017; 8(1):1-3) was examined for identification of the predicted amino acids composing the GRP78 epitopes binding to B4 mAb or F6 mAb. This structure was selected because it is the most complete experimentally-determined structure (spans aa 25-633) of H. sapiens GRP78. (PDB ID #6ASY, Yang et al., 2017). FIG. 3 depicts visualization of major residues for antibody binding, with exemplary major and other residues involved in binding shown by arrows. According to the analyzed structure, all of the amino acids of the predicted epitope are contained in alpha-helices or as part of a linker between two alpha-helices and can be separated by alpha-helix (major residues according to shotgun mutagenesis are underlined): (i) K113; (ii) R261, H265, K268, K271, K272; (iii) R279; and, (iv) E329, D333.

### Example 3 Generation of Edited GRP78 Cell Line

A strategy was developed for producing and purifying antibodies from mammalian cells in which the wild-type target of the antibody is a protein that is generally found intracellularly (e.g., in the membrane of an organelle) but which is present on the cell surface in a pathological state in a patient, e.g., on cancer cells or cells present in patients with an autoimmune disease. The method was exemplified with GRP78 and the exemplary anti-GRP78 antibodies described in Examples 1 and 2.

A. Identification of Mutations in Target Protein to Abrogate Antibody Binding

With knowledge of the epitopes involved in antibody binding, mutagenesis was used to modify one or more of the residues to abrogate binding of the produced antibodies while retaining the structure and function of the protein. For the epitope residues identified for the anti-GRP78 antibodies in Table E3, the mutation(s) in GRP78 were chosen to (1) replace one or more of the charged residues with non-charged residues; (2) ensure that the newly selected residues remain solvent-exposed; and (3) ensure that the alpha-helices are conserved in order to prevent secondary structure changes and the possibility of changes in tertiary and quaternary structure to preserve endogenous function and avoid cell death. Formation of alpha-helices is more likely with amino acids with high propensities to form this secondary structure. Furthermore, polar or charged amino acids are more likely to remain solvent-exposed due to their interaction with water; however, polar amino acids are preferred for mutagenesis because charged residues would be more likely to form interactions with the antibodies. The polar uncharged amino acids from highest to lowest propensity to form an alpha-helix (according to helical penalty) are glutamine (ranked 6th), serine (ranked 10th), asparagine (ranked 15th), and threonine (ranked 16th) (Pace and Scholtz, Biophysical journal. 1998; 75(1):422-7). Glutamine was chosen as the amino acid to replace the proposed amino acids interacting with the mAbs. The replacement of major interacting amino acids with alanine (ranked 1st for alpha-helical propensity) (Pace and Scholtz, 1998) would be more likely to abrogate binding to mAbs albeit with a risk of altering secondary and tertiary structures due to its hydrophobic R group. Thus, it would be more strategic to incorporate alanine less often but at the most critical sites.

Table E4 identifies GRP78 mutations for abrogating binding to the exemplary B4 and F6 mAb.

TABLE E4

| Abrogated Binding to | Abrogated Binding to | Abrogated Binding to |
|---|---|---|
| B4 mAb and F6 mAb | B4 mAb | F6 mAb |
| R261Q + H265Q + | R261Q + H265Q + | R261Q + R279Q |
| R279Q + E329Q | E329Q | |
| R261Q + H265Q + | R261Q + H265Q + | K113Q + R261Q + |
| K271Q + R279Q + | K271Q + K272Q + | K268Q + K271Q + |
| E329Q | E329Q + D333Q | R279Q |
| K113Q + R261Q + | R261A + H265A + | R261A + R279A |
| H265Q + K268Q + | E329A | |
| K271Q + K272Q + | | |
| R279Q + E329Q + | | |
| D333Q | | |
| R261A + H265A + | | |
| R279A + E329A | | |

The proposed GRP78 mutants were modeled using Phyre2 (intensive processing) (Kelley et al., Nature protocols. 2015; 10(6):845-58). Full-length GRP78 sequences (wild-type and ten mutants) were inputted. The wild-type sequence was modeled as a control. Confidence was listed as 100% for residues ˜25-633 for all eleven generated structures. Residues ˜1-24 and ˜634-654, which are not important for the analysis, were modeled ab initio.

Each of the Phyre2-generated mutant GRP78 structures were compared to the Phyre2-generated wild-type GRP78 structure via the Matchmaker function of UCSF Chimera (Pettersen et al., Journal of computational chemistry. 2004; 25(13):1605-12). The wild-type structure was utilized as the reference for this feature. In comparison to the wild-type structure, the regions containing the mutations in all mutant structures were visually unchanged and no alterations in alpha-helices were noted. Nonetheless, root-mean-square deviation (RMSD) values were calculated according to pruned Cα atoms (654 possible pairs) as shown in Table E5:

TABLE E5
Structure analysis of GRP78 mutants

| | RMSD | Pruned C_{α} |
|---|---|---|
| Human GRP78 Mutant | (Å) | Pairs (No.) |
| R261Q + H265Q + R279Q + E329Q | 0.341 | 532 |
| R261Q + H265Q + K271Q + R279Q + E329Q | 0.360 | 525 |
| K113Q + R261Q + H265Q + K268Q + K271Q + | 0.355 | 549 |
| K272Q + R279Q + E329Q + D333Q | | |
| R261A + H265A + R279A + E329A | 0.270 | 541 |
| R261Q + H265Q + E329Q | 0.294 | 543 |
| R261Q + H265Q + K271Q + K272Q + E329Q + | 0.370 | 549 |
| D333Q | | |
| R261A + H265A + E329A | 0.261 | 535 |
| R261Q + R279Q | 0.509 | 581 |
| K113Q + R261Q + K268Q + K271Q + R279Q | 0.515 | 589 |
| R261A + R279A | 0.397 | 555 |

In summary, the proposed GRP78 mutants involve the replacement of residues that interact with B4 mAb and/or F6 mAb (all of which were charged amino acids) with glutamine (polar uncharged amino acid with greatest propensity to form alpha-helix) or alanine (amino acid with overall greatest propensity to form alpha-helix though hydrophobicity). According to modeling, these mutants ensure the conservation of secondary and tertiary structures of GRP78 which simultaneously promotes the retention of native function of GRP78 and the abrogation of binding to the aforementioned mAbs.

The single mutation R261Q was chosen for subsequent experiments as detailed in Section B below as being a major residue of both the B4 and F6 mAb. In addition, the double mutation R261Q/H265Q also was chosen for subsequent experiments as detailed in Section B below, as this double mutant cell modifies two residues identified as part of its epitope, and were mutations found in the combinations that abrogated binding as shown in Table E4. The combination of mutations in the double mutant were specifically selected because 1) they alter residues that were determined by epitope mapping to be potentially important for epitope binding of the GFR78 antibody to native intracellular protein and 2) the mutations are close enough to fit in the same DNA donor template for CRISPR-based editing. Other combinations could also have been used and would have required multiple donor templates and/or transfections be used to introduce the mutations into the cells, either simultaneously or in sequence.

B. Editing the Binding Epitope in the Target Protein

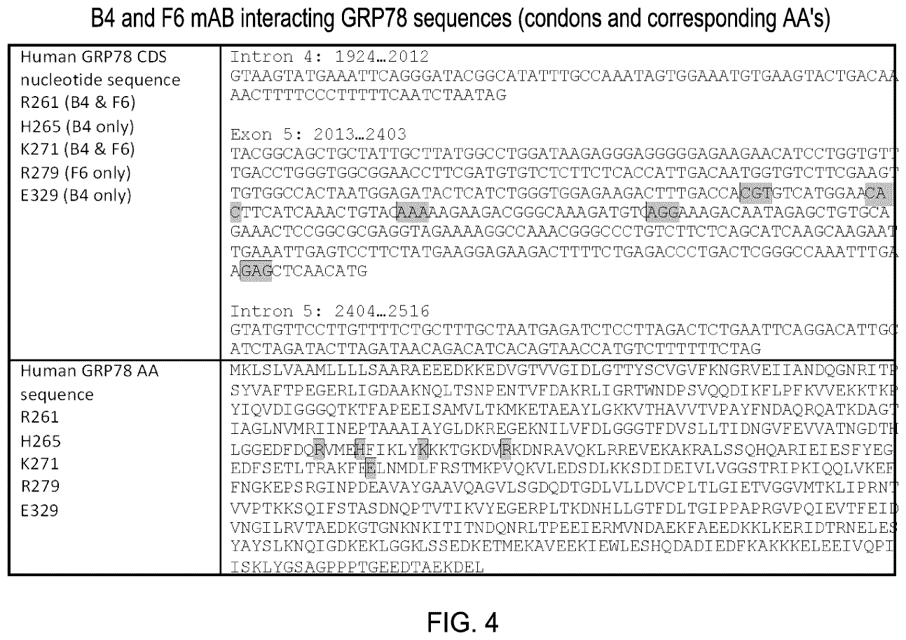

Genomic sequence data for GRP78 was obtained from the NCBI Gene database (Gene ID: 3309) and was used as the reference sequence for gene editing. For both B4 mAb and F6 mAb, the codons of the predicted interacting residues on GRP78 are located within Exon5, as shown in FIG. 4. Flanking introns also are shown in FIG. 4.

A homology-directed repair (HDR) strategy was used to edit wild-type GRP78 (SEQ ID NO:25) using the CRISPR/Cas9 technology to generate the single mutant GRP78 with mutation R261Q (SEQ ID NO:26) or the double mutant GRP78 with mutations R261Q/H265Q (SEQ ID NO:27). See, e.g., Zhang et al., U.S. Pat. No. 9,822,372 issued Nov. 21, 2017.

The vector, gRNA and donor template used were the same for the generation of the single mutant and the double mutant R261Q/H265Q clones, and individual clones were identified that contained either the single mutation R261Q or the double R261Q/H265Q mutation. The donor template, although containing two mutations, resulted in some cells with a single mutation and some cells with the double mutation.

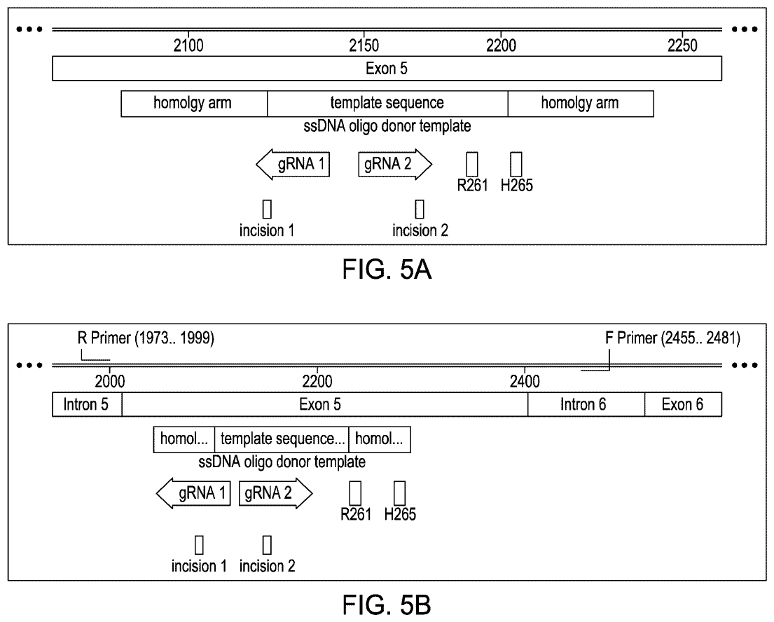

The R261Q single mutation cells and R261Q/H265Q double mutation cells were made by introduction of genomic nucleotide modifications at position 2188-2190 in Exon 5 of CGT→CAG (converting R to Q) and genomic nucleotide modifications at position 2200-2202 in Exon 5 of CAC→CAG (converting H to Q) (FIG. 4). gRNA and ssDNA donor template were designed for introducing the mutations by an HDR-based CRISPR gene editing (Table E6). The dual gRNAs were designed in a “PAM-out” orientation as shown in FIG. 5A. The modified codons in the ssDNA oligo donor sequence to effect the genomic modifications are depicted by bold and italics in Table E6. Nucleotides of the ssDNA were designed to remove the gRNA-associated PAM sites from the donor template, preventing incision of the donor template DNA while conserving the sequence to encode the wild-type amino acids (noted by underline in Table E6).

PCR amplification using the primer pairs set forth in Table E7 were used to validate the gene editing (FIG. 5B). The resulting amplicon is 509 base pairs in length.

TABLE E6
Dual gRNA and ssDNA oligo donor sequence for HDR-based gene editing of GRP78

| | gRNA sequence | gRNA position/ | |
|---|---|---|---|
| gRNA | (SEQ ID NO) | orientation* | incision site |
| 1 | TTCGAAGACACCATTGTCAA | upstream/PAM-out | 2119/2120 |
| | (SEQ ID NO: 28) | | |
| 2 | CACTAATGGAGATACTCATC | downstream/PAM- | 2161/2162 |
| | (SEQ ID NO: 29) | out | |

| | |
|---|---|
| ssDNA oligo | CTGGGTGGCGGAACCTTCGATGTGTCTCTTCTCACGATTGACAATG |
| sequence | GTGTCTTCGAAGTTGTGGCCACTAATGGAGATACTCATCTTGGTGG |
| | AGAAGACTTTGACCAG [symbol] GTCATGGAA [symbol] TTCATCAAACTGTAC |
| | AAAAAGAAGACGGGCAAAGATGTCA (SEQ ID NO: 30) |

TABLE E7
Primers for amplification of region of interest within the GRP78 gene

| | Primer sequence (SEQ ID NO) | Primer binding region |
|---|---|---|
| forward primer | TCTAAGTATCTAGATGCAATGTCCTGA | 2455...2481 |
| | (SEQ ID NO: 31) | |
| reverse primer | TGAAGTACTGACAAAACTTTTCCCTTT | 1973...1999 |
| | (SEQ ID NO:32) | |

Cells harboring the desired mutations were isolated by clonal serial dilution and the presence of point mutations was confirmed by Sanger Sequencing. After expansion in culture, cells were tested for growth and viability. Control (HEK293), GRP78 R261Q (single mutation) and GRP78 R261Q H265Q (double mutation) cells reached >95% confluence at the same time (day 4 post seeding), indicating no alterations in cell growth or viability. Cell confluence was calculated using the IncuCyte® live cell analysis system.

C. Production and Purification of Anti-Gprp78 Antibodies in Modified Hek293 Cells

The fully human anti-GRP78 monoclonal antibodies (mAb) formatted as full-length IgG1 antibodies described in Table E1 were individually transfected into the clonal HEK293 cells by electroporation in 6-well plates, topped up with fresh media the following day and incubated for 5 days post-transfection. Transfection was carried out using the Neon™ transfection system and Neon™ transfection system reagents (Thermo Fisher Scientific, Waltham, MA) using pre-set settings for HEK293 cells. HEK293 wild-type cells were used as control. Presence of human IgG in the supernatant of cells was measure by ELISA.

Results shown in Table E8 demonstrate that the clonal cell populations carrying the GRP78 single mutant R261Q or the double mutations R261Q H265Q can produce and secrete anti-GRP78 IgG antibodies that recognize the epitope containing the native amino acids in substantially higher amounts than the antibodies when produced from HEK293 wild-type cells. Of note, antibodies to GRP78 that do not recognize this epitope are not able to be produced in any significant amount in these cells, as production of these antibodies results in significant reduction of cell viability (data not shown).

Clonal lines were adapted to grow in serum-free media and in suspension. Suspension cultures in serum-free media are used for full scale antibody production. Briefly, cells were passaged 2× in increasing concentrations of serum free media (FreeStyle™ 293 Expression Medium, ThermoFisher Scientific, Waltham, MA). Concentrations for dilution were, in order, 25% serum-free medium+75% (DMEM+10% FBS); 50% serum-free medium+50% (DMEM+10% FBS); and 75% serum-free medium+25% (DMEM+10% FBS) and finally 100% serum-free medium. The cells were diluted into suspension flasks and passaged following pelleting at 125 rpm in an 8% CO₂ 37° C. humidified incubator. Adaptation and growth were monitored daily by cell counting and viability. Cells were considered adapted to suspension when viability following passaging was 95% or higher and doubling time was approximately 24-26 h.

TABLE E8
Antibody Production in HEK293 Clonal Cells

| | |
|---|---|
| Anti-GRP78 (F6) | Anti-GRP78 (B4) |

| | | | | |
|---|---|---|---|---|
| HEK293 WT (control) | 550 | ng/mL | 240 | ng/mL |
| GRP78 R261Q | 1,000 | ng/mL | 9,000 | ng/mL |

| | | | |
|---|---|---|---|
| GRP78 R261Q H265Q | Not measured | 6,000 | ng/mL |

### Example 4 Generation of Cell Lines for Production of Humanized Antibodies Against Signaling Proteins

A. Targeting of Intracellular Signaling Proteins

Proteins involved in intracellular signaling, such as kinases and phosphatases, may also become extracellular during cancer progression and are promising targets for various forms of immunotherapies. The production of antibodies against these targets can be enhanced using the methods and systems of the present disclosure. For example, the protein “phosphatase of regenerating liver 3” (or “PRL-3”, also known as “PTP4A3”) is a member of the PRL family of dual-specificity protein tyrosine phosphatases (Zeng Q et al., Biochem Biophys Res Commun. 1998; 244(2):421-427.). PRL-3 is localized to the cytoplasmic face of the plasma membrane and endosomes via its prenylated C-termini (Zeng Q et al., J Biol Chem. 2000; 275(28):21444-21452).

PRL-3 was identified as a metastasis-associated phosphatase, e.g., with specific upregulation in metastatic colorectal cancer (Saha S, et al. Science. 2001; 294(5545):1343-1346), and elevated PRL-3 expression was shown to be a significant predictor of metastatic recurrence in uveal melanoma patients (Laurent C, et al. Cancer Res. 2011; 71(3):666-674). Elevated PRL-3 mRNA expression levels have clinically been shown to correlate with higher metastatic potential and poor prognosis of multiple cancer types, including gastric cancer (Bessette D C, et al., Cancer Metastasis Rev. 2008; 27(2):231-252). Since then, PRL-3 has been reported to be overexpressed in up to 70% of primary gastric carcinomas, with higher PRL-3 expression correlating with shorter postoperative survival at all tumor stages in gastric cancer patients (Li Z R, et al. Surg Today. 2007; 37(8):646-651; Dai Net al., World J Gastroenterol. 2009; 15(12):1499-1505).

It has been shown that intracellular PRL-3 antigens are externalized during the cancer process to become extracellular, and thus PRL-3 is a potential target for immunotherapy to treat various forms of cancer that exhibit extracellular PRL-3 expression. The production methods and system of the invention are thus useful for the production of humanized or human PRL-3 antibodies for use as human therapeutics.

B. Generation of Edited Prl3 Cell Lines

A humanized anti-PRL3 antibody was engineered from the original framework of a previously characterized murine anti-PRL-3 antibody as described in Thura M, et al. (JCI Insight. 2016; 1(9):e87607). Briefly, the humanized anti-PRL-3 antibody (dubbed “PRL3-zumab”) was engineered from the original framework of the previously characterized murine an anti-PRL-3 antibody clone. The CDRs of the heavy and light chains of the mouse antibody were grafted onto human sequence frameworks, which were chosen by aligning the mouse framework sequences against a database of human framework sequences to find the closest human homolog for each chain. In addition to the CDRs from the mouse sequence, three amino acid positions from the mouse sequence flanking the CDRs were also grafted into the human acceptor sequence to preserve the original murine anti-PRL-3 antibody's CDR structural integrity.

### Example 5 Generation of Cell Line for Anti-PRL-3 Humanized Antibody Purification

A. Identification of Mutations in the Target Protein to Abrogate Antibody Binding

The epitope of PRL3-zumab was shown to specifically recognize an epitope within a C-terminal region conserved between both mouse and human PRL-3, but not PRL-1 or PRL-2. Id. With knowledge of the epitope being within the C-terminal region of PRL-3, in conjunction with the crystal structure of PRL-3 (as shown by Kim K A et al., FEBS Lett (2004) 565 p.181-7), mutagenesis is used to modify one or more of the residues in this conserved C-terminal region of the native PRL-3 protein in the production cell lines to abrogate binding of the produced antibodies while retaining the overall structure and function of the protein.

Specifically, the mutation(s) in native mammalian PRL-3 C-terminal binding region is chosen to conserve the structure of the protein to prevent secondary structure changes and the possibility of changes in tertiary and quaternary structure to preserve endogenous function and avoid cell death. The replacement of amino acids of PRL-3 with alanine at critical sites is predicted and proposed PRL-3 mutants in mammalian cells evaluated further for viability and abrogation of binding of the humanized mAbs to the intracellular protein. Depending on the cell line chosen for antibody production, the sequence modified can be based on the sequence of the epitope of the species of the cell line, although the epitope is generally conserved between mammalian species such as rodents and humans.

B. Editing the Binding Epitope in the Target Protein

The gene sequence data for PRL-3 in humans (Gene ID: 11156) and the corresponding sequence for PRL-3 from Chinese hamsters (Gene ID: 100772907) are obtained from the NCBI Gene database and used as reference sequences for gene editing in HEK293 cells and CHO cells, respectively. For production of anti-PRL-3 antibody, a homology-directed repair (HDR) strategy is used to edit wild-type PRL-3 in the HEK293 cells (SEQ ID NO:33) and CHO cells (SEQ ID NO:34) using Type V CRISPR gene editing technology to generate the desired single and/or double mutant. Such editing strategies are found in Garst et al, U.S. Pat. No. 11,220,697 issued Jan. 11, 2022, and in “Gene Editing with MAD7™ in mammalian cells: Quick Start Guide” which can be found at www.inscripta.com. In addition, tools for design of specific guide RNAs for use with Type V RGNs can be found from companies such as Integrated DNA Technologies (Coralville, IA) and GenScript (Piscataway, NJ).

Cells harboring the desired mutations are isolated by clonal serial dilution and the presence of point mutations are confirmed by Sanger Sequencing. After expansion in culture, cells are tested for growth and viability. Control (wtCHO), and mutant PRL-3 CHO cells are tested, and cells that reach >90% confluence at the same time (day 4 post seeding) are selected as exhibiting minimal alterations in cell growth or viability. Cell confluence is calculated using the IncuCyte® live cell analysis system.

C. Production and Purification of Anti-Prl3 Antibodies in Modified Cells

The humanized anti-PRL-3 monoclonal antibodies (mAb) are individually transfected into the clonal CHO cells by electroporation in 6-well plates, topped up with fresh media the following day and incubated for 5 days post-transfection. Wild-type HEK293 or CHO cells are used as controls. The presence of human IgG in the supernatant of cells is measured by ELISA. Cells that produce and secrete anti-PRL-3 IgG antibodies in substantially higher amounts than the antibodies when produced from HEK293 and/or CHO wild-type cells are selected.

### Example 6 Humanization of an Anti-PDI Monoclonal Mouse Antibody

The disclosed methods for producing and purifying antibodies from mammalian cells as disclosed in Example 3 is also useful for production of mAbs that selectively bind to other intracellular proteins, and such methods can not only increase production by enhancing cell viability but can also increase the amount of recoverable antibody by allowing the antibody to be secreted or otherwise available for purification rather than bound to intracellular targets. The methods are particularly useful for production of antibodies that selectively bind to intracellular mammalian targets, including those in, e.g., the ER and the Golgi. The following examples disclose the production of the humanized antibodies against an ER target, protein disulfide isomerase (PDI).

PDI, also known as the beta-subunit of prolyl 4-hydroxylase, is an enzyme that in humans encoded by the P4HB gene. PDI acts as a chaperone that catalyzes disulfide bond formation, breakage and rearrangement in the endoplasmic reticulum (ER). Increasing evidence supports a key role for PDI in maintaining cellular homeostasis by mediating oxidative protein folding. The progression of several cancers, and in particular brain cancers such as glioblastoma, have been linked to increased expression of PDI, and PDI inhibitors have been investigated for treatment of cancers such as glioblastoma. See, e.g., Kyani et al., ChemMedChem, 2018 January 22; 13(2):164-177. PDI inhibition has been demonstrated to downregulate DNA repair pathways and genes in the E2F pathway in glioblastoma cells, and thus PDI remains a very promising oncology target. Xu S., et al., Theranostics. 2019; 9(8): 2282-2298.

The Invitrogen PDI Monoclonal Antibody Clone 12 (Thermo Fisher Scientific Catalog #MA5-43389, Thermo Fisher Scientific, Waltham, MA), hereafter referred to as “mPDI-1” is a mouse IgG1 monoclonal antibody that selectively binds to human PDI/P4HB (“hPDI”). Invitrogen PDI Monoclonal Antibody clone 2F6G12H2 (Thermo Fisher Scientific Catalog #MA5-43389, Thermo Fisher Scientific, Waltham, MA), hereafter referred to as “mPDI-2”, is distinct mouse IgG1 monoclonal antibody that selectively binds to hPDI. Both of these antibodies were selected using a recombinant human PDI protein as an antigen in a mouse host, and both are sensitive enough for use in ELISA assays; thus, both are potential candidates for humanization to create a therapeutic monoclonal antibody targeting hPDI. Because PDI is found primarily in the ER, the methods of the present disclosure are well suited for efficient production of such antibodies in mammalian cells.

The humanized PDI antibodies for use in the present methods preferably comprise CDR sequences derived from or based on mPDI-1 or mPDI-2, as described in more detail herein and in the incorporated references. These antibodies can be humanized as taught in more detail herein.

Briefly, the amino acid sequences of the V_{H} and V_{L} regions of mPDI-1 and mPDI-2 are determined using the REmAB® antibody sequencing services provided by Rapid Novor (Ontario, Canada). The CDRs of mPDI-1 and mPDI-2 can be determined using the IMGT numbering system as provided, e.g., in Lefranc, M.-P. et al., Dev. Comp. Immunol., 27, 55-77 (2003). Once the CDRs are identified, the mouse monoclonal antibodies can be humanized using methods such as those disclosed in, e.g., Do Couto et al., U.S. Pat. No. 10,613,094, issued Apr. 7, 2020, and Chilcote et al., U.S. Pat. No. 8,673,593, issued Mar. 18, 2014.

### Example 7 Epitope Mapping of the Protein Binding Site of the Anti-PDI Humanized Antibodies to hPDI

Various methods can be employed for epitope mapping of the binding site of mPDI-1 and mPDI-2 mAbs on human hPDI. Exemplary methods are taught in U.S. Pat. No. 11,174,479 to Greenleaf, et al., issued on Nov. 16, 2021, and U.S. Pat. No. 11,061,036 to Wilson, et al., issued on Jul. 13, 2021.

For example, the binding of mPDI-1 and mPDI-2 mAbs to biotinylated PDI peptides that span hPDI can be determined using the Streptavidin biosensors. Biotinylated peptides spanning the hPDI protein are loaded at 5 μg/ml for 700 seconds, 300 seconds for baseline recording and then mPDI-1 or mPDI-2 association and dissociation with each of the peptides measured for 600 seconds each at multiple concentrations. Double reference sensors are employed in all testing to measure and subtract any observed background signal from nonspecific binding or system noise.

Analysis is performed with ForteBio Data Analysis Software (v8.2). After background subtraction, a 1:1 local kinetic model is fit to the observed association and dissociation curves. Overall, K_{D}, Kₒₙ, K_{off}, and R2 correlation coefficients are determined. Where possible, a global curve fit is also performed for multiple concentrations of the antibody/analyte.

### Example 8 Identification of hPDI Structure to Define Key Residues for Mutagenesis

To identify the residues to be edited to abrogate binding of the antibodies to hPDI, a combination of the crystal structure of the S. cerevisiae (Tian G., et al., Cell. 2006; 124:61-73) and the crystallization of human PDI in both the oxidized and reduced states (Wang C, et al., Antioxid Redox Signal 19: 36-45) can be used to extrapolate to the structure and function of the human PDI. Large scale molecular dynamics simulations starting from the crystal structures of human PDI (hPDI) in the oxidized and reduced states demonstrate the intrinsic conformational dynamics. PDI is composed of four thioredoxin-like domains, and the protein can form at least four compact confirmations. Yang S. et al., PLoS One. 2014; 9(8): e103472.

### Example 9 Generation of Cell Line for Anti-PDI Humanized Antibody Purification

A. Identification of Mutations in the Target Protein to Abrogate Antibody Binding

With knowledge of the epitopes involved in binding of the humanized mAbs based on mPDI-1 and mPDI-2 to hPDI, and the crystal structures and molecular modeling of the hPDI protein, mutagenesis is used to modify one or more of the residues to abrogate binding of the produced antibodies while retaining the overall structure and function of the protein. The mutation(s) in native mammalian PDI are chosen to conserve the structure of the protein to prevent secondary structure changes and the possibility of changes in tertiary and quaternary structure to preserve endogenous function and avoid cell death. The replacement of amino acids of PDI with alanine at critical sites is predicted and proposed PDI mutants in mammalian cells evaluated further for viability and abrogation of binding of the humanized mAbs based on mPDI-1 and mPDI-2 to the intracellular protein.

The proposed PDI mutants are modeled using Phyre2 (intensive processing) (Kelley et al., Nature protocols. 2015; 10(6):845-58). Full-length PDI sequences (wild-type and ten mutants) are inputted, and the wild-type sequence is modeled as a control. Each of the Phyre2-generated mutant PDI structures are compared to the Phyre2-generated wildtype PDI structure via the Matchmaker function of UCSF Chimera (Pettersen et al., Journal of computational chemistry. 2004; 25(13):1605-12). The wild-type structure of hPDI is utilized as the reference for this feature. In comparison to the wild-type structure, the regions containing the mutations in the desirable mutant structures are visually unchanged with no significant alterations in secondary structure.

B. Editing the Binding Epitope in the Target Protein

Genomic sequence data for PDI in Chinese hamster cells is obtained from the NCBI Gene database (Gene ID:100766687) and is used as the reference sequence for gene editing. For production of both PDI-1 mAb and PDI-2 mAb, a homology-directed repair (HDR) strategy was used to edit wild-type PDI in the CHO cells (SEQ ID NO:34) using Type V CRISPR gene editing technology to generate the desired single and/or double mutant. Such editing strategies are found in Garst et al, U.S. Pat. No. 11,220,697 issued Jan. 11, 2022, and in “Gene Editing with MAD7™ in mammalian cells: Quick Start Guide” which can be found at www.inscripta.com. In addition, tools for design of specific guide RNAs for use with Type V RGNs can be found from companies such as Integrated DNA Technologies (Coralville, IA) and GenScript (Piscataway, NJ).

Cells harboring the desired mutations are isolated by clonal serial dilution and the presence of point mutations are confirmed by Sanger Sequencing. After expansion in culture, cells are tested for growth and viability. Control (wtCHO), and mutant PDI CHO cells are tested, and cells that reach >90% confluence at the same time (day 4 post seeding) are selected as exhibiting minimal alterations in cell growth or viability. Cell confluence is calculated using the IncuCyte® live cell analysis system.

C. Production and Purification of Anti-Hpdi Antibodies in Modified Cho Cells

The humanized anti-hPDI monoclonal antibodies (mAb) formatted as full-length IgG1 antibodies are individually transfected into the clonal CHO cells by electroporation in 6-well plates, topped up with fresh media the following day and incubated for 5 days post-transfection. Wild-type CHO cells are used as control. Presence of human IgG in the supernatant of cells is measured by ELISA. Cells that produce and secrete anti-hPDI IgG antibodies in substantially higher amounts than the antibodies when produced from CHO wild-type cells are selected

### Example 10 Humanization of an Anti-GOLPH3 Monoclonal Mouse Antibody

GOLPH3 was initially identified as a peripheral membrane protein localized to the trans-Golgi network, but others reported it to be a mitochondrial protein that regulated the mitochondrial mass through the regulation of the mitochondria-specific phospholipid cardiolipin. GOLPH3 has since been implicated in the target of rapamycin (TOR) signaling pathway. GOLPH3-transfected cells enhanced S6 Kinase activity in response to growth factor stimulation by EGF. Simultaneously, AKT phosphorylation increased in these cells, while these events were abrogated in GOLPH3 siRNA treated cells compared to control cells, indicating the GOLPH3 can enhance signaling through TOR-associated complexes. These results suggest that GOLPH3 is a bona fide oncogene and may be a useful target for therapeutic strategies.

The Thermo Fisher Monoclonal Antibody Clone 905CT9.1.1 (Thermo Fisher Scientific Catalog #MA5-37626, Thermo Fisher Scientific, Waltham, MA), hereafter referred to as “mGOLPH3” is a mouse IgG1 monoclonal antibody that selectively binds to human purified His-tagged GOLPH3 protein. Because GOLPH3 is found primarily in the Golgi and the mitochondria, the methods of the present disclosure are well suited for efficient production of such antibodies in mammalian cells.

The humanized GOLPH3 antibodies for use in the present methods preferably comprise CDR sequences derived from or based on mGOLPH3, as described in more detail herein and in the incorporated references. Briefly, the amino acid sequences of the V_{H} and V_{L} regions of mGOLPH3 are determined using the REmAB® antibody sequencing services provided by Rapid Novor (Ontario, Canada). The CDRs of mGOLPH3 can be determined using the IMGT numbering system as provided, e.g., in Lefranc, M.-P. et al., Dev. Comp. Immunol., 27, 55-77 (2003). Once the CDRs are identified, the mouse monoclonal antibodies can be humanized using methods such as those disclosed in, e.g., Do Couto et al., U.S. Pat. No. 10,613,094, issued Apr. 7, 2020, and Chilcote et al., U.S. Pat. No. 8,673,593, issued Mar. 18, 2014.

### Example 11 Epitope Mapping of the Protein Binding Site of the Anti-GOLPH3 Humanized Antibodies to hGOLPH3

Various methods can be employed for epitope mapping of the binding site of the anti-mGOLPH3 mAb on human hGOLPH3. Exemplary methods are taught in U.S. Pat. No. 11,174,479 to Greenleaf, et al., issued on Nov. 16, 2021, and U.S. Pat. No. 11,061,036 to Wilson, et al., issued on Jul. 13, 2021.

For example, the binding of mGOLPH3 mAb to biotinylated hGOLPH3 peptides that span hGOLPH3 can be determined using the Streptavidin biosensors. Biotinylated peptides spanning the hGOLPH3 protein are loaded at 5 μg/ml for 700 seconds, 300 seconds for baseline recording and then mGOLPH3 mAb association and dissociation with each of the peptides measured for 600 seconds each at multiple concentrations. Double reference sensors are employed in all testing to measure and subtract any observed background signal from nonspecific binding or system noise.

Analysis is performed with ForteBio Data Analysis Software (v8.2). After background subtraction, a 1:1 local kinetic model was fit to the observed association and dissociation curves. Overall, K_{D}, Kₒₙ, K_{off}, and R2 correlation coefficients are determined. Where possible, a global curve fit was also performed for multiple concentrations of the antibody/analyte.

### Example 12 Identification of hGOLPH3 Structure to Define Key Residues for Mutagenesis

To identify the residues to be edited to abrogate binding of the antibodies to hGOLPH3, the crystal structure hGOLPH3 and its orthologs can be used. Wood et al. have reported the X-ray crystal structure of hGOLPH3 to a 2.9-Å resolution (Wood et al., J Cell Biol. 2009 Dec. 28; 187(7): 967-975), as well as conserved regions of activity in the structures of hGOLPH3 and its yeast ortholog, Vps74p (Wood et al., Journal of Cell Biology, 209 187:67-75. Additional structural aspects and residues required for specific functions of GOLPH3 can be found in Bergeron J J M et al., Mol Cell Proteomics. 2017 December; 16(12): 2048-2054 and Schmitz K R, et al., Dev. Cell 2008; 14:523-534).

### Example 13 Generation of Cell Lines for Anti-GOLPH3 Humanized Antibody Purification

### A. Identification of Mutations in Target Protein to Abrogate Antibody Binding

With knowledge of the epitopes involved in binding of the humanized mAbs based on the mGOLPH3 mAb to hGOLPH3, and the crystal structures and molecular modeling of the hGOLPH protein, mutagenesis is used to modify one or more of the residues to abrogate binding of the produced antibodies while retaining the overall structure and function of the GOLPH3 protein. The mutation(s) in native mammalian cell GOLPH3 are chosen to conserve the structure of the protein to prevent secondary structure changes and the possibility of changes in tertiary and quaternary structure to preserve endogenous function and avoid negative manufacturing outcomes, e.g., cell death, decreased cell viability and/or reduction in antibody production. The replacement of amino acids of hGOLPH3 with alanine at critical sites is predicted and proposed GOLPH3 mutants in mammalian cells evaluated further for viability and abrogation of binding of the humanized mAbs based on mGOLPH3 mAb to the intracellular protein.

The proposed GOLPH3 mutants are modeled using Phyre2 (intensive processing) (Kelley et al., Nature protocols. 2015; 10(6):845-58). Full-length GOLPH3 sequences (wild-type and ten mutants) are inputted, and the wild-type sequence is modeled as a control. Each of the Phyre2-generated mutant GOLPH3 structures are compared to the Phyre2-generated wild-type GOLPH3 structure via the Matchmaker function of UCSF Chimera (Pettersen et al., Journal of computational chemistry. 2004; 25(13):1605-12). The wild-type structure of hGOLPH3 is utilized as the reference for this feature. In comparison to the wild-type structure, the regions containing the mutations in the desirable mutant structures are visually unchanged with no significant alterations in secondary structure.

### B. Editing the Binding Epitope in the Target Protein

Genomic sequence data for GOLPH3 in Chinese hamster cells is obtained from the NCBI Gene database (Gene ID:100766687) and is used as the reference sequence for gene editing. For production of humanized mGOLPH3 mAb, a homology-directed repair (HDR) strategy was used to edit wild-type GOLPH3 in the CHO cells (SEQ ID NO:36) using the Type V CRISPR gene editing technology to generate the desired mutant (e.g., mutation of one or more residues). Such editing strategies are found in Garst et al, U.S. Pat. No. 11,220,697 issued Jan. 11, 2022, and in “Gene Editing with MAD7™ in mammalian cells: Quick Start Guide” which can be found at www.inscripta.com. In addition, tools for design of specific guide RNAs for use with Type V RGNs can be found from companies such as Integrated DNA Technologies (Coralville, IA) and GenScript (Piscataway, NJ).

Cells harboring the desired mutations are isolated by clonal serial dilution and the presence of point mutations are confirmed by Sanger Sequencing. After expansion in culture, cells are evaluated for growth and viability. Control (wtCHO), and mutant GOLPH3 CHO cells are tested, and cells that reach >90% confluence at the same time (day 4 post seeding) are selected as exhibiting minimal alterations in cell growth or viability. Cell confluence is calculated using the IncuCyte® live cell analysis system.

### C. Production and Purification of Anti-Golph3 Antibodies in Modified Cho Cells

The humanized anti-GOLPH3 monoclonal antibodies (mAb) formatted as full-length IgG1 antibodies are individually transfected into the clonal CHO cells by electroporation in 6-well plates, topped up with fresh media the following day and incubated for 5 days post-transfection. Wild-type CHO cells are used as control. Presence of human IgG in the supernatant of cells is measured by ELISA. Cells that produce and secrete anti-hGOLPH3 IgG antibodies in substantially higher amounts than the antibodies when produced from CHO wild-type cells are selected.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| # | SEQUENCE | ANNOTATION |
|---|---|---|
| 1 | GGSISSGGYY | B4 VH CDR1 |
| 2 | YIYYSGSTYY NPSLES | B4 VH CDR2 |
| 3 | RYSSIDAFEI | B4 VH CDR3 |
| 4 | GGDDIGSKSV H | B4 VL CDR1 |
| 5 | DDGDRPS | B4 VL CDR2 |
| 6 | QVWDSSSDQY V | B4 VL CDR3 |
| 7 | GDSVSSNSAA | D1 VH CDR1 |
| 8 | RTYYRSRWYN DYAVSVES | D1 VH CDR2 |
| 9 | DPYYYDSSGY YYFDAFGI | D1 VH CDR3 |
| 10 | GGNNIGSKSV H | D1 VL CDR1 |
| 11 | DGSDRPS | D1 VL CDR2 |
| 12 | DDSDRPS | D1 VL CDR3 |
| 13 | GDSVSSNSAA | F6 VH CDR1 |
| 14 | RTYYRSKWYN DYAVSVKS | F6 VH CDR2 |
| 15 | DPYYYDSSGY YYFDAFDI | F6 VH CDR3 |
| 16 | GGDNIGSKSV H | F6 VL CDR1 |
| 17 | DDSDRPS | F6 VL CDR2 |
| 18 | QVWDSTSHHV V | F6 VL CDR3 |
| 19 | QVRLQESGPG LVKPSQTLSL TCTVSGGSIS SGGYYWSWIR | B4 VH |
| | QHPGKGLEWI GYIYYSGSTY YNPSLESRVT ISVDTSKNQF | |
| | SLKLSSVTAA DTAVYYCARY | |
| | SSIDAFEIWG QGTMVTVSS | |
| 20 | SYVLTQPPSV SVAPGKTATI TCGGDDIGSK SVHWYQQKPG | B4 VL |
| | QAPVLVVYDD GDRPSGIPER FSGSNSGNTA TLAISRVEAG | |
| | DEADYYCQVW DSSSDQYVFG | |
| | SGTKLTVL | |
| 21 | QVQLQQSGPG LVEPSQTLSL TCAISGDSVS SNSAAWNWIR | D1 VH |
| | QSPSRGLEWL GRTYYRSRWY NDYAVSVESR ITINPDTSKN | |
| | QFSLQLNSVT PEDTAVYYCA RDPYYYDSSG YYYFDAFGIW | |
| | GQGTMVTVSS | |
| 22 | SYELIQPPSV SVAPGQTARI ACGGNNIGSK SVHWYQQKPG | D1 VL |
| | QAPVLVVYDD SDRPSGIPER FSGSNSGNTA TLTISRVEAG | |
| | DEADYYCQVW DSSSDHPGVF GTGTKLTVL | |
| 23 | QVQLQQSGPG LVKPPQTLSL TCAISGDSVS SNSAAWNWIR | F6 VH |
| | QSPSRGLEWL GRTYYRSKWY NDYAVSVKSR ITINPDTSKN | |
| | QFSLQLNSVT PEDTAVYYCA RDPYYYDSSG YYYFDAFDIW | |
| | GQGTMVTVSS | |
| 24 | SYELTQPHSV SVAPGQTARI TCGGDNIGSK SVHWYQQRPG | F6 VL |
| | QAPVLVVYDD SDRPSGIPER FSGSNSENTA TLTISGVEAG | |
| | DEADYYCQVW DSTSHHVVFG GGTKLTVL | |
| 25 | MKLSLVAAMLLLLSAARAEEEDKKEDVGTVVGIDLGTTYSCV | Wild-Type |
| | GVFKNGRVEIIANDQGNRITPSYVAFTPEGERLIGDAAKNQLTS | Human GRP78 |
| | NPENTVFDAKRLIGRTWNDPSVQQDIKFLPFKVVEKKTKPYIQ | |
| | VDIGGGQTKTFAPEEISAMVLTKMKETAEAYLGKKVTHAVVT | |
| | VPAYFNDAQRQATKDAGTIAGLNVMRIINEPTAAAIAYGLDK | |
| | REGEKNILVFDLGGGTFDVSLLTIDNGVFEVVATNGDTHLGGE | |
| | DFDQRVMEHFIKLYKKKTGKDVRKDNRAVQKLRREVEKAKR | |
| | ALSSQHQARIEIESFYEGEDFSETLTRAKFEELNMDLFRSTMKP | |
| | VQKVLEDSDLKKSDIDEIVLVGGSTRIPKIQQLVKEFFNGKEPS | |
| | RGINPDEAVAYGAAVQAGVLSGDQDTGDLVLLDVCPLTLGIE | |
| | TVGGVMTKLIPRNTVVPTKKSQIFSTASDNQPTVTIKVYEGER | |
| | PLTKDNHLLGTFDLTGIPPAPRGVPQIEVTFEIDVNGILRVTAE | |
| | DKGTGNKNKITITNDQNRLTPEEIERMVNDAEKFAEEDKKLKE | |
| | RIDTRNELESYAYSLKNQIGDKEKLGGKLSSEDKETMEKAVEE | |
| | KIEWLESHQDADIEDFKAKKKELEEIVQPIISKLYGSAGPPPTG | |
| | EEDTAEKDEL | |
| 26 | MKLSLVAAMLLLLSAARAEEEDKKEDVGTVVGIDLGTTYSCV | R261Q GRP78 |
| | GVFKNGRVEIIANDQGNRITPSYVAFTPEGERLIGDAAKNQLTS | |
| | NPENTVFDAKRLIGRTWNDPSVQQDIKFLPFKVVEKKTKPYIQ | |
| | VDIGGGQTKTFAPEEISAMVLTKMKETAEAYLGKKVTHAVVT | |
| | VPAYFNDAQRQATKDAGTIAGLNVMRIINEPTAAAIAYGLDK | |
| | REGEKNILVFDLGGGTFDVSLLTIDNGVFEVVATNGDTHLGGE | |
| | DFDQQVMEHFIKLYKKKTGKDVRKDNRAVQKLRREVEKAKR | |
| | ALSSQHQARIEIESFYEGEDFSETLTRAKFEELNMDLFRSTMKP | |
| | VQKVLEDSDLKKSDIDEIVLVGGSTRIPKIQQLVKEFFNGKEPS | |
| | RGINPDEAVAYGAAVQAGVLSGDQDTGDLVLLDVCPLTLGIE | |
| | TVGGVMTKLIPRNTVVPTKKSQIFSTASDNQPTVTIKVYEGER | |
| | PLTKDNHLLGTFDLTGIPPAPRGVPQIEVTFEIDVNGILRVTAE | |
| | DKGTGNKNKITITNDQNRLTPEEIERMVNDAEKFAEEDKKLKE | |
| | RIDTRNELESYAYSLKNQIGDKEKLGGKLSSEDKETMEKAVEE | |
| | KIEWLESHQDADIEDFKAKKKELEEIVQPIISKLYGSAGPPPTG | |
| | EEDTAEKDEL | |
| 27 | MKLSLVAAMLLLLSAARAEEEDKKEDVGTVVGIDLGTTYSCV | R261Q + |
| | GVFKNGRVEIIANDQGNRITPSYVAFTPEGERLIGDAAKNQLTS | H265Q GRP78 |
| | NPENTVFDAKRLIGRTWNDPSVQQDIKFLPFKVVEKKTKPYIQ | |
| | VDIGGGQTKTFAPEEISAMVLTKMKETAEAYLGKKVTHAVVT | |
| | VPAYFNDAQRQATKDAGTIAGLNVMRIINEPTAAAIAYGLDK | |
| | REGEKNILVFDLGGGTFDVSLLTIDNGVFEVVATNGDTHLGGE | |
| | DFDQQVMEQFIKLYKKKTGKDVRKDNRAVQKLRREVEKAKR | |
| | ALSSQHQARIEIESFYEGEDFSETLTRAKFEELNMDLFRSTMKP | |
| | VQKVLEDSDLKKSDIDEIVLVGGSTRIPKIQQLVKEFFNGKEPS | |
| | RGINPDEAVAYGAAVQAGVLSGDQDTGDLVLLDVCPLTLGIE | |
| | TVGGVMTKLIPRNTVVPTKKSQIFSTASDNQPTVTIKVYEGER | |
| | PLTKDNHLLGTFDLTGIPPAPRGVPQIEVTFEIDVNGILRVTAE | |
| | DKGTGNKNKITITNDQNRLTPEEIERMVNDAEKFAEEDKKLKE | |
| | RIDTRNELESYAYSLKNQIGDKEKLGGKLSSEDKETMEKAVEE | |
| | KIEWLESHQDADIEDFKAKKKELEEIVQPIISKLYGSAGPPPTG | |
| | EEDTAEKDEL | |
| 28 | TTCGAAGACACCATTGTCAA | gRNA 1 |
| 29 | CACTAATGGAGATACTCATC | gRNA 2 |
| 30 | CTGGGTGGCGGAACCTTCGATGTGTCTCTTCTCACGATTGA | ssDNA oligo |
| | CAATGGTGTCTTCGAAGTTGTGGCCACTAATGGAGATACTC | sequence |
| | ATCTTGGTGGAGAAGACTTTGACCAGCAGGTCATGGAACA | |
| | GTTCATCAAACTGTACAAAAAGAAGACGGGCAAAGATGTC | |
| | A | |
| 31 | TCTAAGTATCTAGATGCAATGTCCTGA | Forward primer |
| 32 | TGAAGTACTGACAAAACTTTTCCCTTT | Reverse primer |
| 33 | MARMNRPAPVEVSYKHMRFLITHNPTNATLSTFIEDLKKYGA | Wild-type |
| | TTVVRVCEVTYDKTPLEKDGITVVDWPFDDGAPPPGKVVED | human PRL-3 |
| | WLSLVKAKFCEAPGSCVAVHCVAGLGRAPVLVALALIESGM | |
| | KYEDAIQFIRQKRRGAINSKQLTYLEKYRPKQRLRFKDPHTHK | |
| | TRCCVM | |
| 34 | MARMNRPAPVEVSYRNMRFLITHNPSNATLSTFIEDLKKYGA | Wild-type |
| | TTVVRVCEVTYDKTPLEKDGITVVDWPFDDGAPPPGKVVED | Chinese |
| | WLSLLKAKFYNDPGSCVAVHCVAGLGRAPVLVALALIESGM | Hamster PRL-3 |
| | KYEDAIQFIRQKRRGAINSKQLTYLEKYRPKQRLRFKDPHTHK | |
| | TRCCVM | |
| 35 | MLSRSLLCLALAWVARVGADAPEEEDNVLVLKKSNFAEALA | Wild-type |
| | AHNYLLVEFYAPWCGHCKALAPEYAKAAAKLKAEGSEIRLA | Chinese |
| | KVDATEESDLAQQYGVRGYPTIKFFKNGDTASPKEYTAGREA | Hamster |
| | DDIVNWLKKRTGPAATTLSDTAAAETLIDSSEVAVIGFFKDVE | PDI |
| | SDSAKQFLLAAEAVDDIPFGITSNSGVFSKYQLDKDGVVLFKK | |
| | FDEGRNNFEGEVTKEKLLDFIKHNQLPLVIEFTEQTAPKIFGGEI | |
| | KTHILLFLPKSVSDYDGKLGNFKKAAEGFKGKILFIFIDSDHTD | |
| | NQRILEFFGLKKEECPAVRLITLEEEMTKYKPESDELTAEKITE | |
| | FCHRFLEGKIKPHLMSQELPEDWDKQPVKVLVGKNFEEVAFD | |
| | EKKNVFVEFYAPWCGHCKQLAPIWDKLGETYKDHENIIIAKM | |
| | DSTANEVEAVKVHSFPTLKFFPATADRTVIDYNGERTLDGFKK | |
| | FLESGGQDGAGDDDDVDLEEALEPDMEEDDDQKAVKDEL | |
| 36 | MTSLTQRSSGLVQRRTEASRNAADKERAAGGGGGSGEDEAQ | Wild-type |
| | SRRDEQDDDDKGDSKETRLTLMEEVLLLGLKDREGYTSFWN | Chinese |
| | DCISSGLRGCMLIELALRGRLQLEACGMRRKSLLTRKVVCKSD | Hamster |
| | APTGDVLLDEALKHVKETQPPETVQNWIELLSGETWNPLKLH | GOLPH3 |
| | YQLRNVRERLAKNLVEKGVLTTEKQNFLLFDMTTHPLTNNNI | |
| | KQRLIKKVQEAVLDKWVNDPHRMDKRLLALIYLAHASDVLE | |
| | NAFAPLLDEQYDLATKRVRQLLDLDPEVECLKANTNEVLWA | |
| | VVAAFTK | |

1. A method of recombinant production of an antibody, the method comprising:
(a) expressing a nucleic acid encoding an antibody in a modified cell line, wherein the antibody specifically binds to a target epitope on a target protein that is natively expressed as an intracellular protein in the cell; and wherein the modified cell is engineered to express a variant target protein that comprises a mutation of one or more amino acid residues of the target epitope; and
(b) culturing the modified cell line under conditions so that the antibody is produced.

2. The method of claim 1, further comprising (c) isolating the antibody from the culture supernatant.

3. The method of claim 1, wherein the target protein that is natively expressed is an intracellular protein in the secretory pathway of the cell line.

4. The method of claim 1, wherein the mutation is an inert mutation that retains the structure and function of the native target protein in the cell.

5. The method of claim 1, wherein the mutation reduces or abrogates binding of the antibody to the target epitope on the target protein.

6. The method of claim 1, wherein the target protein is a stress protein.

7. The method of claim 1, wherein the target protein is expressed on an organelle of the cell line.

8. The method of claim 7, wherein the organelle is the endoplasmic reticulum or the Golgi apparatus.

9. The method of claim 1, wherein the target protein is an endoplasmic reticulum chaperone.

10. The method of claim 9, wherein the endoplasmic reticulum chaperone is a calreticulin, a heat shock protein or an isomerase.

11. The method of claim 1, wherein the target protein is glucose-regulated protein 78 (GRP78), HSP47, PDI, calreticulin or GP94.

12. The method of claim 1, wherein the target protein is glucose-regulated protein 78 (GRP78).

13. The method of claim 1, wherein the target protein is a Golgi complex protein.

14. The method of claim 13, wherein the Golgi complex protein is GOLPH2, GOLPH3, GM130, ATP6V1A, ATP6V1E1, ATP6VOA2, TMEM165, GOLGB1, SCYL1BP1, TRAPPC11, TRAPPC2 or TRIP11.

15. A method of recombinant production of an antibody, the method comprising:
(a) expressing a nucleic acid encoding an antibody in a modified mammalian cell line, wherein the antibody specifically binds a target epitope on a target protein that is natively expressed as an intracellular protein in the mammalian cell line and wherein the modified mammalian cell line is engineered to express a variant target protein that comprises a mutation of one or more amino acid residues of the target epitope; and
(b) culturing the modified mammalian cell line under conditions so that the antibody is produced.

16. The method of claim 15, further comprising (c) isolating the antibody from the culture supernatant.

17. The method of claim 15, wherein the target protein that is natively expressed is an intracellular protein in the secretory pathway of the mammalian cell line.

18. The method of claim 15, wherein the mutation is an inert mutation that retains the structure and function of the target protein in the mammalian cell line.

19. The method of claim 15, wherein the mutation reduces or abrogates binding of the antibody to the target epitope on the target.

20. The method of claim 15, wherein the target protein is a stress protein.

21. The method of claim 15, wherein the target protein is expressed on an organelle of the mammalian cell line.

22. The method of claim 21, wherein the organelle is the endoplasmic reticulum or the Golgi apparatus.

23. The method of claim 15, wherein the target protein is an endoplasmic reticulum chaperone.

24. The method of claim 23, wherein the endoplasmic reticulum chaperone is a calreticulin, a heat shock protein or an isomerase.

25. The method of claim 15, wherein the target protein is glucose-regulated protein 78 (GRP78), HSP47, PDI, calreticulin or GP94.

26. The method of claim 15, wherein the target protein is glucose-regulated protein 78 (GRP78).

27. The method of claim 15, wherein the target protein is a Golgi complex protein.

28. The method of claim 27, wherein the Golgi complex protein is GOLPH2, GOLPH3, GM130, ATP6V1A, ATP6V1E1, ATP6VOA2, TMEM165, GOLGB1, SCYL1BP1, TRAPPC11, TRAPPC2 or TRIP11.

29. A method of recombinant production of an antibody targeting an epitope on a target protein found intracellularly in a cell line, the method comprising:
(a) providing an antibody that binds to an epitope on a target protein that is present intracellularly in a cell line;
(b) identifying the epitope of the target protein to which the antibody binds;
(c) generating a modified cell line by mutating the epitope of the target protein in the cell line to reduce or abrogate the binding of the antibody to the epitope on the target protein;
(d) introducing an expression vector encoding the antibody to the modified cell line;
(e) culturing the modified cell line under conditions that allow production of the antibody from the expression vector; and
(f) expressing a nucleic acid encoding an antibody in the modified cell line.

30. The method of claim 29, wherein the produced antibody is secreted to the culture supernatant.

31. The method of claim 29, further comprising (g) isolating the antibody or antigen binding fragment.

32. The method of claim 29, wherein the antibody is a humanized antibody.

33. The method of claim 29, wherein the antibody is a human antibody.

34. The method of claim 29, wherein the target protein that is present intracellularly in the cell line is an intracellular protein in the secretory pathway of the cell line.

35. The method of claim 29, wherein the target protein is a stress protein.

36. A method of recombinant production of an antibody targeting an epitope on a target protein found intracellularly in a mammalian cell line, the method comprising:
(a) providing an antibody that binds to an epitope on a target protein that is present intracellularly in a mammalian cell line;
(b) identifying the epitope of the target protein to which the antibody binds;
(c) generating a modified cell line by mutating the epitope of the target protein in the mammalian cell line to reduce or abrogate the binding of the antibody to the epitope on the target protein;
(d) introducing an expression vector encoding the antibody to the modified mammalian cell line;
(e) culturing the modified mammalian cell line under conditions that allow production of the antibody from the expression vector; and
(f) expressing a nucleic acid encoding an antibody in the modified mammalian cell line.

37. The method of claim 36, wherein the produced antibody is present in the culture supernatant.

38. The method of claim 37, further comprising (g) isolating the antibody or antigen binding fragment.

39. The method of claim 36, wherein the antibody is a humanized antibody.

40. The method of claim 36, wherein the antibody is a human antibody.

41. The method of claim 29, wherein the target protein that is present intracellularly in the mammalian cell line is an intracellular protein in the secretory pathway of the mammalian cell line.

42. The method of claim 29, wherein the target protein is a stress protein.

43. A method of recombinant production of an antibody targeting GRP78, the method comprising:
(a) expressing a nucleic acid encoding an antibody in a modified mammalian cell line, wherein the antibody binds to GRP78 and wherein the modified mammalian cell is engineered with a variant GRP78 that has a mutant epitope comprising a mutation of one or more amino acid residues of the target epitope of the antibody or antigen binding fragment; and
(b) culturing the modified mammalian cell line under conditions so that the anti-GRP78 antibody is produced in the culture supernatant.

44. The method of claim 43, further comprising (c) isolating the anti-GRP78 antibody from the culture supernatant.

45. The method of claim 43, wherein the antibody is a humanized antibody.

46. The method of claim 43, wherein the antibody is a human antibody.

47. A modified mammalian cell line comprising a variant protein that is an intracellular protein in the secretory pathway, wherein the variant protein comprises a mutation of a native intracellular protein that is a target protein of an antibody and wherein the mutation is an amino acid substitution(s) of one or more amino acid residues to change a target epitope of the antibody to a mutant epitope.

48. The modified mammalian cell line of claim 47, wherein the mutation is an inert mutation that retains the structure and function of the target protein of the mammalian cell.

49. The modified mammalian cell line of claim 47, wherein the mutation reduces or abrogates binding of the antibody to the variant protein.

50. The modified mammalian cell line of claim 47, wherein the target protein is a stress protein.

51. A modified mammalian cell line comprising a variant GRP78 comprising the sequence set forth in SEQ ID NO:28.

52. A modified mammalian cell line comprising a variant GRP78 comprising the sequence set forth in SEQ ID NO:29.

53. The modified mammalian cell line of claim 47, wherein the mammalian cell line is selected from the group consisting of Chinese hamster ovary (CHO) cells, Baby hamster kidney cells, NSO myeloma cells, monkey kidney COS cells, monkey kidney fibroblast CV-I cells, human embryonic kidney 293 (HEK293) cells, human breast cancer SKBR3 cells, human leukemia Jurkat T cells, dog kidney MDCK cells, and human cervical cancer HeLa cells.

54. A method of recombinant production of an antibody, the method comprising:
(a) expressing a nucleic acid encoding an antibody in a modified mammalian cell line, wherein the antibody specifically binds a target epitope on an intracellular signaling protein that is Response to Notice to File Missing Parts and Notice of Incomplete Reply natively expressed in the mammalian cell line and wherein the modified mammalian cell line is engineered to express a variant intracellular signaling protein that comprises a mutation of one or more amino acid residues of the target epitope; and
(b) culturing the modified mammalian cell line under conditions so that the antibody is produced.

55. The method of claim 54, further comprising (c) isolating the antibody from the culture supernatant.

56. The method of claim 54, wherein intracellular signaling protein is a phosphatase.

57. The method of claim 54, wherein intracellular signaling protein is a kinase.


SEQUENCE LISTING (36 sequences)

SEQ ID NO: 1 - protein, 10 aa, Homo sapiens
GGSISSGGYY

SEQ ID NO: 2 - protein, 16 aa, Homo sapiens
YIYYSGSTYYNPSLES

SEQ ID NO: 3 - protein, 10 aa, Homo sapiens
RYSSIDAFEI

SEQ ID NO: 4 - protein, 11 aa, Homo sapiens
GGDDIGSKSVH

SEQ ID NO: 5 - protein, 7 aa, Homo sapiens
DDGDRPS

SEQ ID NO: 6 - protein, 11 aa, Homo sapiens
QVWDSSSDQYV

SEQ ID NO: 7 - protein, 10 aa, Homo sapiens
GDSVSSNSAA

SEQ ID NO: 8 - protein, 18 aa, Homo sapiens
RTYYRSRWYNDYAVSVES

SEQ ID NO: 9 - protein, 18 aa, Homo sapiens
DPYYYDSSGYYYFDAFGI

SEQ ID NO: 10 - protein, 11 aa, Homo sapiens
GGNNIGSKSVH

SEQ ID NO: 11 - protein, 7 aa, Homo sapiens
DGSDRPS

SEQ ID NO: 12 - protein, 7 aa, Homo sapiens
DDSDRPS

SEQ ID NO: 13 - protein, 10 aa, Homo sapiens
GDSVSSNSAA

SEQ ID NO: 14 - protein, 18 aa, Homo sapiens
RTYYRSKWYNDYAVSVKS

SEQ ID NO: 15 - protein, 18 aa, Homo sapiens
DPYYYDSSGYYYFDAFDI

SEQ ID NO: 16 - protein, 11 aa, Homo sapiens
GGDNIGSKSVH

SEQ ID NO: 17 - protein, 7 aa, Homo sapiens
DDSDRPS

SEQ ID NO: 18 - protein, 11 aa, Homo sapiens
QVWDSTSHHVV

SEQ ID NO: 19 - protein, 119 aa, Homo sapiens
QVRLQESGPGLVKPSQTLSLTCTVSGGSISSGGYYWSWIRQHPGKGLEWIGYIYYSGSTYYNPSLESRVTISVDTSKNQFSLKLSSVTAADTAVYYCARYSSIDAFEIWGQGTMVTVSS

SEQ ID NO: 20 - protein, 108 aa, Homo sapiens
SYVLTQPPSVSVAPGKTATITCGGDDIGSKSVHWYQQKPGQAPVLVVYDDGDRPSGIPERFSGSNSGNTATLAISRVEAGDEADYYCQVWDSSSDQYVFGSGTKLTVL

SEQ ID NO: 21 - protein, 130 aa, Homo sapiens
QVQLQQSGPGLVEPSQTLSLTCAISGDSVSSNSAAWNWIRQSPSRGLEWLGRTYYRSRWYNDYAVSVESRITINPDTSKNQFSLQLNSVTPEDTAVYYCARDPYYYDSSGYYYFDAFGIWGQGTMVTVSS

SEQ ID NO: 22 - protein, 109 aa, Homo sapiens
SYELIQPPSVSVAPGQTARIACGGNNIGSKSVHWYQQKPGQAPVLVVYDDSDRPSGIPERFSGSNSGNTATLTISRVEAGDEADYYCQVWDSSSDHPGVFGTGTKLTVL

SEQ ID NO: 23 - protein, 130 aa, Homo sapiens
QVQLQQSGPGLVKPPQTLSLTCAISGDSVSSNSAAWNWIRQSPSRGLEWLGRTYYRSKWYNDYAVSVKSRITINPDTSKNQFSLQLNSVTPEDTAVYYCARDPYYYDSSGYYYFDAFDIWGQGTMVTVSS

SEQ ID NO: 24 - protein, 108 aa, Homo sapiens
SYELTQPHSVSVAPGQTARITCGGDNIGSKSVHWYQQRPGQAPVLVVYDDSDRPSGIPERFSGSNSENTATLTISGVEAGDEADYYCQVWDSTSHHVVFGGGTKLTVL

SEQ ID NO: 25 - protein, 654 aa, Homo sapiens
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 26 - protein, 654 aa, Synthetic construct
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQQVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 27 - protein, 654 aa, synthetic construct
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQQVMEQFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 28 - DNA, 20 bp, Synthetic construct
  source 1..20: mol_type = other DNA
ttcgaagacaccattgtcaa

SEQ ID NO: 29 - DNA, 20 bp, Synthetic construct
  source 1..20: mol_type = other DNA
cactaatggagatactcatc

SEQ ID NO: 30 - DNA, 163 bp, Synthetic construct
  source 1..163: mol_type = other DNA
ctgggtggcggaaccttcgatgtgtctcttctcacgattgacaatggtgtcttcgaagttgtggccactaatggagatactcatcttggtggagaagactttgaccagcaggtcatggaacagttcatcaaactgtacaaaaagaagacgggcaaagatgtca

SEQ ID NO: 31 - DNA, 27 bp, Synthetic construct
  source 1..27: mol_type = other DNA
tctaagtatctagatgcaatgtcctga

SEQ ID NO: 32 - DNA, 27 bp, Synthetic construct
  source 1..27: mol_type = other DNA
tgaagtactgacaaaacttttcccttt

SEQ ID NO: 33 - protein, 173 aa, Homo sapiens
MARMNRPAPVEVSYKHMRFLITHNPTNATLSTFIEDLKKYGATTVVRVCEVTYDKTPLEKDGITVVDWPFDDGAPPPGKVVEDWLSLVKAKFCEAPGSCVAVHCVAGLGRAPVLVALALIESGMKYEDAIQFIRQKRRGAINSKQLTYLEKYRPKQRLRFKDPHTHKTRCCVM

SEQ ID NO: 34 - protein, 173 aa, Phodopus sp.
MARMNRPAPVEVSYRNMRFLITHNPSNATLSTFIEDLKKYGATTVVRVCEVTYDKTPLEKDGITVVDWPFDDGAPPPGKVVEDWLSLLKAKFYNDPGSCVAVHCVAGLGRAPVLVALALIESGMKYEDAIQFIRQKRRGAINSKQLTYLEKYRPKQRLRFKDPHTHKTRCCVM

SEQ ID NO: 35 - protein, 509 aa, Phodopus sp.
MLSRSLLCLALAWVARVGADAPEEEDNVLVLKKSNFAEALAAHNYLLVEFYAPWCGHCKALAPEYAKAAAKLKAEGSEIRLAKVDATEESDLAQQYGVRGYPTIKFFKNGDTASPKEYTAGREADDIVNWLKKRTGPAATTLSDTAAAETLIDSSEVAVIGFFKDVESDSAKQFLLAAEAVDDIPFGITSNSGVFSKYQLDKDGVVLFKKFDEGRNNFEGEVTKEKLLDFIKHNQLPLVIEFTEQTAPKIFGGEIKTHILLFLPKSVSDYDGKLGNFKKAAEGFKGKILFIFIDSDHTDNQRILEFFGLKKEECPAVRLITLEEEMTKYKPESDELTAEKITEFCHRFLEGKIKPHLMSQELPEDWDKQPVKVLVGKNFEEVAFDEKKNVFVEFYAPWCGHCKQLAPIWDKLGETYKDHENIIIAKMDSTANEVEAVKVHSFPTLKFFPATADRTVIDYNGERTLDGFKKFLESGGQDGAGDDDDVDLEEALEPDMEEDDDQKAVKDEL

SEQ ID NO: 36 - protein, 298 aa, Phodopus sp.
MTSLTQRSSGLVQRRTEASRNAADKERAAGGGGGSGEDEAQSRRDEQDDDDKGDSKETRLTLMEEVLLLGLKDREGYTSFWNDCISSGLRGCMLIELALRGRLQLEACGMRRKSLLTRKVVCKSDAPTGDVLLDEALKHVKETQPPETVQNWIELLSGETWNPLKLHYQLRNVRERLAKNLVEKGVLTTEKQNFLLFDMTTHPLTNNNIKQRLIKKVQEAVLDKWVNDPHRMDKRLLALIYLAHASDVLENAFAPLLDEQYDLATKRVRQLLDLDPEVECLKANTNEVLWAVVAAFTK